(19) United States
(12) Patent Application Publication
(10) Pub. No.: US 2026/0284150 A1
(43) Pub. Date: Sep. 24, 2026

(54) TOPICAL PROTEIN BASED FORMULATION

(71) Applicant: OPTIMUS SALVUS LIMITED, Gibraltar (GB)

(72) Inventors: Stella VALENZUELA, New South Wales (AU); Khondker Rufaka HOSSAIN, New South Wales (AU); Joanne PATTERSON, New South Wales (AU)

(21) Appl. No.: 19/475,891

(22) PCT Filed: Apr. 18, 2024
(86) PCT No.: PCT/AU2024/050372

§ 371 Date: Oct. 16, 2025

(30) Foreign Application Priority Data
Apr. 18, 2023 (AU) 2023901143

(51) Int. Cl. A61K 38/44; A61K 9/127; A61K 31/00; A61P 39/06

(52) U.S. Cl. CPC A61K 38/44; A61K 9/127; A61K 31/658; A61P 39/06; C12Y 108/05001

(57) ABSTRACT

The present disclosure generally relates to transdermal formulations comprising one or more chloride intracellular channel (CLIC) proteins in combination with one or more cannabinoid compounds, processes for preparing the transdermal formulations, and applications for the transdermal formulations.

### TECHNICAL FIELD

The present disclosure generally relates to transdermal formulations comprising one or more chloride intracellular channel (CLIC) proteins and one or more cannabinoid compounds, processes for preparing the transdermal formulations, and applications for the transdermal formulations.

### BACKGROUND

Mammalian skin is composed of three distinct layers: the epidermis, dermis, and hypodermis. The epidermis, the most outer layer skin, is composed of five different horizontal layers: the stratum basale, stratum spinosum, stratum granulosum, stratum lucidum, and stratum corneum. Within the epidermis, new keratinocytes are formed and replace previously shed layers. The keratinocytes are accompanied by melanocytes, which serve to produce the photoprotective skin pigment melanin. Combined, this protective section of the skin allows for continuous shedding of cells by friction, which is replaced by newly formed cells in the deeper sections of the epidermis and serves as a UV protective barrier.

Oxidative stress plays a major role in the aging process, this consists of both intrinsic and extrinsic factors. Intrinsic ageing of the skin is a natural inevitable occurrence of physiological change over time. These rates are often invariable and genetically predetermined. Extrinsic factors, on the other hand, are more controllable to different degrees. Such factors include exposure to sunlight, pollution, diet, repetitive muscle movements, nicotine/tobacco use, and overall health. Sunlight on the skin alone (UVA, UVB, and UVC radiation) is estimated to account for approximately 90% of visible skin ageing. Both intrinsic and extrinsic ageing factors can work in synergy over the human lifetime to deteriorate the skin barrier with drastic effects. Aged skin will become susceptible to dryness, itching, infection, autoimmunity, vascular complication, and increase risks of malignancy. These ageing factors may cause fine wrinkles due to the reduction of collagen, elastic fibres, and hyaluronic acid.

Despite clear damage to collagen synthesis, mitochondria are especially vulnerable to oxidative damage due to high exposure to levels of reactive oxygen species.

Therefore, developing new transdermal formulations, that may deliver improved or enhanced skin health effects, is of interest and in demand.

Consequently, there is a need to provide alternative or improved transdermal formulations and processes for preparing alternative or improved transdermal formulations for use in skin care, cosmetics, medical and pharmaceutical applications.

### SUMMARY

The present inventors have prepared transdermal formulations comprising one or more chloride intracellular channel (CLIC) proteins, one or more cannabinoid compounds, and a carrier. The present inventors have also identified a process for preparing transdermal formulations comprising one or more chloride intracellular channel (CLIC) proteins, one or more cannabinoid compounds, and a carrier. It has been found that formulations prepared by the processes defined herein provide alternative or improved stable transdermal formulations comprising one or more chloride intracellular channel (CLIC) proteins, one or more cannabinoid compounds, and a carrier. One or more advantages of the present disclosure, according to at least some embodiments or examples as described herein, is that a transdermal formulation can be prepared from one or more chloride intracellular channel (CLIC) proteins, one or more cannabinoid compounds, and a carrier, wherein the CLIC proteins in combination with at least one cannabinoid compound may provide protective effects when administered as a topical formulation to skin of a subject. This may be demonstrated by increased cell viability. It will be appreciated that chloride intracellular channel (CLIC) proteins have previously been shown to behave as atypical ion channels.

In one aspect there is provided a transdermal formulation comprising one or more CLIC proteins and one or more cannabinoid compounds, in a therapeutically and/or prophylactically effective amount, and a carrier. In some embodiments, the CLIC protein may be in an amount between about 0.0001% and 10% based on total weight of the formulation. In some embodiments, the CLIC protein may be selected from the group comprising or consisting of CLIC1, CLIC2, CLIC3, CLIC4, CLIC5, CLIC6, or combinations thereof. In some embodiments, the weight ratio of CLIC protein to carrier may be between about 1:1, 1:4, 1:5, 1:10, 1:20, 1:25, 1:50, 1:100, 1:1000, 1:10000 or 1:100000.

In some embodiments, the one or more cannabinoid compounds may be selected from the group consisting of cannabidiol (CBD), cannabidiol A (CBDA) cannabigerol (CBG), cannabichromene (CBC), cannabicyclol (CBL), cannabinol (CBN), cannabigerolic acid (CBGA), cannabidivarin (CBDV), cannabichromevarin (CBCV), cannabigerovarin (CBGV), cannabigerol monomethyl ether (CBGM), or a combination thereof. In one example, the cannabinoid compound may be cannabidiol (CBD). In some embodiments, the cannabinoid compound may be present in the form of a cannabinoid extract or isolate having less than 1, 0.5, or 0.3 wt % of any of tetrahydrocannabinol (THC), tetrahydrocannabivarin (THCV) and tetrahydrocannabinolic acid (THCA). The formulation may comprise the one or more cannabinoid compounds in a concentration between about 0.0001 mg/ml to about 1200 mg/ml.

In some embodiments, the carrier may be a solvent carrier. The solvent carrier may be an aqueous soluble solvent carrier. In some embodiments, the solvent carrier may be selected from the group comprising water, alcohols, lanolin alcohols, fatty alcohols, glycols, oils, waxes, phospholipids, sterols, isopropyl myristate, oleic acid, or combinations thereof. The solvent carrier may be in an amount between about 2% and 99.99% based on total weight of the formulation.

In some embodiments, the formulation may comprise one or more optional additives. The optional additive may be selected from the group comprising antioxidant, preservative, salt, sugar, sugar alcohol, polysaccharide, vitamin, essential oil, rheology modifier, transdermal compounding base or gel, lipoic acid, xanthan gum, surfactant, or any combination thereof. In some embodiments, the additive may be present in an amount between about 0.01% and 15% based on total weight of the formulation.

In some embodiments, the formulation may further comprise an optional penetration enhancer. The penetration enhancer may be selected from the group comprising water, sulphoxide, azone, pyrrolidone, alcohol, alkanol, glycol, surfactant, fatty acid, terpene, terpenoid, or any combination thereof. In some embodiments, the penetration enhancer may be present in an amount between about 0.01% and 15% based on total weight of the formulation.

In some embodiments, the formulation may further comprise an optional buffer selected from the group comprising glycerol, glutathione, phosphate-based buffer, acetate buffer, phosphate buffer, citrate buffer, TRIS buffer, maleate buffer, succinate buffer, histidine buffer, amino acids, or combinations thereof. The buffer may be in an amount between about 0.01% and 15% based on total weight of the formulation.

In some embodiments, the formulation may be in the form of an aqueous solution, alcohol solution, ointment, lotion, gel, paste, salve, controlled-release matrix, transdermal delivery system, or any combination thereof.

In another aspect there is provided a method of reducing and/or preventing oxidative stress in cells of mammalian skin, the method comprising: topically administering a transdermal formulation to the mammalian skin, wherein the transdermal formulation comprises a therapeutically and/or prophylactically effective amount of one or more CLIC proteins, one or more cannabinoid compounds, and a carrier.

In another aspect there is provided a method of rejuvenating UV damaged cells in mammalian skin, the method comprising: topically administering a transdermal formulation to the mammalian skin, wherein the transdermal formulation comprises a therapeutically effective amount of one or more CLIC proteins, one or more cannabinoid compounds, and a carrier.

In yet another aspect there is provided a method of treating UV damaged cells and/or preventing UV damage in mammalian skin, the method comprising: topically administering a transdermal formulation to the mammalian skin, wherein the transdermal formulation comprises a therapeutically and/or prophylactically effective amount of one or more CLIC proteins, one or more cannabinoid compounds, and a carrier.

In some embodiments, the methods may comprise steps of administering the formulation topically in the form of an ointment, cream, lotion, gel, salve, liquid spray, controlled-release matrix, transdermal delivery system, patch formulation, or any combination thereof.

In some embodiments, the methods may comprise steps of administering the formulation over a time period of about 1 day to about 6 months.

In some embodiments, the methods may comprise steps of administering the formulation in a dosage of the one or more CLIC proteins in a range between about 0.0001 mg to about 100 mg per day and the one or more cannabinoid compounds in a range between about 0.001 mg to about 100 mg per day.

In some embodiments, the methods may comprise steps of administering the formulation one, two, three or four times per day.

In some embodiments, the mammalian skin, or cells therein, may be that of a human patient in need of such treatment or prophylaxis.

In another aspect there is provided a use of a CLIC protein selected from the group consisting of: CLIC1, CLIC2, CLIC3, CLIC4, CLIC5, CLIC6, and combinations thereof, in combination with one or more cannabinoid compounds, in the manufacture of a transdermal medicament for the reduction of oxidative stress in cells of mammalian skin and/or for the prevention of oxidative stress to said cells of mammalian skin.

In another aspect there is provided a use of a CLIC protein selected from the group consisting of: CLIC1, CLIC2, CLIC3, CLIC4, CLIC5, CLIC6, and combinations thereof, in combination with one or more cannabinoid compounds, in the manufacture of a transdermal medicament for the rejuvenation of UV damaged cells in mammalian skin.

In yet another aspect there is provided a use of a CLIC protein selected from the group consisting of: CLIC1, CLIC2, CLIC3, CLIC4, CLIC5, CLIC6, and combinations thereof, in combination with one or more cannabinoid compounds, in the manufacture of a transdermal medicament for the treatment of UV damaged cells of mammalian skin and/or for the prevention of UV damage to said cells of mammalian skin.

In another aspect there is provided a process for preparing a transdermal formulation comprising an effective amount of one or more CLIC proteins and a carrier comprising the steps of: combining one or more CLIC proteins, one or more cannabinoid compounds, and a carrier; and adding one or more optional additives, an optional penetration enhancer, and an optional buffer.

### BRIEF DESCRIPTION OF FIGURES

Some embodiments of the present disclosure are described and illustrated herein, by way of example only, with reference to the accompanying Figures in which:

FIG. 1 shows the CBD dosage optimisation and Cell Toxicity assay (A) shows the images of HDF cells after 24 hours of incubation with varying concentration of CBD and (B) shows a bar diagram of the % cell viability of HDF cells after 24 hours of CBD treatment.

FIG. 2 shows the cell proliferation and viability assay (using WST-1 Reagent) comparing % cell viability of HDF (A) and HEK (B) and co-cultured cells (C) that were pre-treated for 24 hours with CLIC1 and CLIC4 in varying ratio combinations (CLIC1:CLIC4) in the presence of 3 μg/mL CBD followed by either 1 hour challenge with 0.2 mM H₂O₂ or 20 mins challenge with UV exposure (2.175×10¹³ (J*m⁻²*sec⁻¹)). Total CLIC protein concentrations were maintained at a final CLIC1 concentration=3 μg/mL and CLIC4=5 μg/mL. CBD concentration used was 3 μg/mL. Data is shown as Mean±STDev (n=3). Asterisk signs shows level of significance with * means significance at p value=0.05, **p value=0.01 and ***p value=0.005.

FIG. 3 shows the cell proliferation and viability assay (using WST-1 Reagent) comparing % cell viability of HDF (A) HEK (B) and co-cultured cells (C) that were pre-treated for 24 hours with varying ratio of CLIC1:CLIC4 in the presence or absence of Aq-CBD followed by 1 hour challenge with 0.2 mM H₂O₂. Total CLIC protein concentrations were maintained at a final CLIC1 concentration=3 ug/mL and CLIC4=5 ug/mL. CBD concentration used was 3 ug/mL. Data is shown as Mean±SEM (n=9). Asterisk signs shows level of significance with * means significance at p value <0.05, **p value <0.01, ***p value <0.005 and p value <0.001.

FIG. 4 shows cell proliferation and viability assay (using WST-1 Reagent) comparing % cell viability of HDF (A) HEK (B) and co-cultured cells (C) that were pre-treated for 24 hours with varying ratio of CLIC1:CLIC4 in the presence or absence of Aq-CBD followed by 20 mins challenge with UV exposure (2.175×1013 (J*m−2*sec−1)). Total CLIC protein concentrations were maintained at a final CLIC1 concentration=3 ug/mL and CLIC4=5 ug/mL. CBD concentration used was 3 ug/mL. Data is shown as Mean±SEM (n=9). Asterisk signs shows level of significance with * means significance at p value <0.05, **p value <0.01, ***p value <0.005 and p value <0.001.

### DETAILED DESCRIPTION

The present disclosure describes the following various non-limiting examples, which relate to investigations undertaken to identify appropriate transdermal formulations comprising one or more chloride intracellular channel (CLIC) proteins and one or more cannabinoid compounds, methods of making, and use thereof. The present inventors have prepared a transdermal formulation comprising one or more chloride intracellular channel (CLIC) proteins, one or more cannabinoid compounds, and a carrier. The present inventors have also identified a process for preparing the transdermal formulation comprising the one or more chloride intracellular channel (CLIC) proteins and one or more cannabinoid compounds.

At least according to some embodiments or examples as described herein, the present disclosure provides a new transdermal formulation comprising one or more CLIC proteins and one or more cannabinoid compounds. The inventors have unexpectedly found that the one or more CLIC proteins in combination with one or more cannabinoid compounds, when administered topically, may provide enhanced cellular enzymatic activity and/or antioxidant activity such as, for example, protection from UV light and oxidative stress when the CLIC protein is overexpressed in skin cells. It has been surprisingly found that enhanced enzymatic activity is an indication of protective effects exhibited by the administration of the transdermal formulation to a subject in need thereof.

### General Definitions and Terms

With regards to the definitions provided herein, unless stated otherwise, or implicit from context, the defined terms and phrases include the provided meanings. In addition, unless explicitly stated otherwise, or apparent from context, the terms and phrases below do not exclude the meaning that the term or phrase has acquired by a person skilled in the relevant art. The definitions are provided to aid in describing particular embodiments, and are not intended to limit the claimed invention, because the scope of the invention is limited only by the claims.

All publications discussed and/or referenced herein are incorporated herein in their entirety.

Unless otherwise required by context, singular terms shall include pluralities and plural terms shall include the singular. Throughout this disclosure, unless specifically stated otherwise or the context requires otherwise, reference to a single step, formulation of matter, group of steps or group of formulations of matter shall be taken to encompass one and a plurality (i.e., one or more) of those steps, formulations of matter, groups of steps or groups of formulations of matter. Thus, as used herein, the singular forms “a”, “an” and “the” include plural aspects unless the context clearly dictates otherwise. For example, reference to “a” includes a single as well as two or more; reference to “an” includes a single as well as two or more; reference to “the” includes a single as well as two or more and so forth.

Those skilled in the art will appreciate that the disclosure herein is susceptible to variations and modifications other than those specifically described. It is to be understood that the disclosure includes all such variations and modifications. The disclosure also includes all of the examples, steps, features, methods, formulations, formulations, and processes, referred to or indicated in this specification, individually or collectively, and any and all combinations or any two or more of said steps or features.

Each example of the present disclosure described herein is to be applied mutatis mutandis to each and every other example unless specifically stated otherwise. The present disclosure is not to be limited in scope by the specific examples described herein, which are intended for the purpose of exemplification only. Functionally-equivalent products, compositions and methods are clearly within the scope of the disclosure as described herein.

The term “and/or”, e.g., “X and/or Y” shall be understood to mean either “X and Y” or “X or Y” and shall be taken to provide explicit support for both meanings or for either meaning.

Unless otherwise indicated, the terms “first,” “second,” etc. are used herein merely as labels, and are not intended to impose ordinal, positional, or hierarchical requirements on the items to which these terms refer. Moreover, reference to a “second” item does not require or preclude the existence of lower-numbered item (e.g., a “first” item) and/or a higher-numbered item (e.g., a “third” item).

As used herein, the phrase “at least one of”, when used with a list of items, means different combinations of one or more of the listed items may be used and only one of the items in the list may be needed. The item may be a particular object, thing, or category. In other words, “at least one of” means any combination of items or number of items may be used from the list, but not all of the items in the list may be required. For example, “at least one of item A, item B, and item C” may mean item A; item A and item B; item B; item A, item B, and item C; item B and item C; or item C. In some cases, “at least one of item A, item B, and item C” may mean, for example and without limitation, two of item A, one of item B, and ten of item C; four of item B and seven of item C; or some other suitable combination.

It is to be appreciated that certain features that are, for clarity, described herein in the context of separate embodiments, may also be provided in combination in a single embodiment. Conversely, various features that are, for brevity, described in the context of a single embodiment, may also be provided separately or in any sub-combination.

Throughout the present specification, various aspects and components of the invention can be presented in a range format. The range format is included for convenience and should not be interpreted as an inflexible limitation on the scope of the invention. Accordingly, the description of a range should be considered to have specifically disclosed all the possible sub-ranges as well as individual numerical values within that range, unless specifically indicated. For example, description of a range such as from 1 to 5 should be considered to have specifically disclosed sub-ranges such as from 1 to 3, from 1 to 4, from 1 to 5, from 2 to 4, from 2 to 5, from 3 to 5 etc., as well as individual and partial numbers within the recited range, for example, 1, 2, 3, 4, 5, 5.5 and 6, unless where integers are required or implicit from context. This applies regardless of the breadth of the disclosed range. Where specific values are required, these will be indicated in the specification.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

Throughout this specification, the term “consists of” is intended to exclude elements which would materially affect the properties of the claimed formulation.

The terms “comprising”, “comprise” and “comprises” herein are intended to be optionally substitutable with the terms “consisting essentially of”, “consist essentially of”, “consists essentially of”, “consisting of”, “consist of” and “consists of”, respectively, in every instance.

As used herein, the term about, unless stated to the contrary, refers to +/−20%, typically +/−10%, typically +/−5%, of the designated value.

As used herein, the term “subject” refers to any organism that is susceptible to a disease or condition. For example, the subject can be an animal, a mammal, a primate, a livestock animal (e.g., sheep, cow, horse, pig), a companion animal (e.g., dog, cat), or a laboratory animal (e.g., mouse, rabbit, rat, guinea pig, hamster). In one example, the subject is a mammal. In one preferred embodiment, the subject is human. In one alternative embodiment, the subject is a non-human animal.

As used herein, the term “treating” includes alleviation of symptoms associated with a specific disease or condition. For example, “treatment” may refer to a therapeutic intervention that ameliorates a sign or symptom of UV damaged cells in mammalian skin.

As used herein, the term “prevention” includes prophylaxis of the specific disease or condition. For example, “preventing” may refer to a therapeutic intervention that prevents oxidative stress in cells of mammalian skin and/or prevents UV damage in mammalian skin.

As used herein, the term “rejuvenating” includes restoring UV damaged cells of mammalian skin to its original form or rejuvenated form.

The term “therapeutically effective amount”, as used herein, refers to a formulation being administered in an amount sufficient to alleviate or prevent to some extent one or more of the symptoms of the disease or condition being treated. The result can be the reduction and/or alleviation of the signs, symptoms, or causes of a disease or condition, or any other desired alteration of a biological system.

The term “prophylactically effective amount”, as used herein, refers to an effective amount of a formulation to prevent or inhibit or delay the onset of a disease or condition.

The term, an “effective amount”, as used herein, refers to an amount of a formulation effective to achieve a desired pharmacologic effect or therapeutic improvement without undue adverse side effects. It is understood that “an effective amount” or “a therapeutically effective amount” can vary from subject to subject, due to variation in metabolism of the compound and any of age, weight, general condition of the subject, the condition being treated, the severity of the condition being treated, and the judgment of the prescribing physician.

Herein “weight %” may be abbreviated to as “wt %”.

### Transdermal Formulations

Formulations suitable for use in the methods and uses described herein comprise a transdermal formulation comprising one or more CLIC proteins and one or more cannabinoid compounds. In some embodiments, the transdermal formulation comprising one or more CLIC proteins and one or more cannabinoid compounds is presented as a topical formulation. In some embodiments, there is provided the use or method as described herein, wherein the transdermal formulation comprising one or more CLIC proteins and one or more cannabinoid compounds, is administered in the form of a topical formulation, e.g. a sunscreen or a cosmetic. The inventors have unexpectedly shown that the one or more CLIC proteins in combination with at least one cannabinoid compound, when administered, may provide enhanced cellular enzymatic activity and/or antioxidant activity such as, for example, protection from UV light and oxidative stress. It has been surprisingly found that increased cell viability is an indication of the protective effects of the combination of CLIC proteins and cannabinoid compounds exhibited by the administration of the transdermal formulation to a subject in need thereof. One or more advantages of the present invention may be provided by the combination of the CLIC proteins with the cannabinoid compounds such that a synergistic effect is observed. For example, when the CLIC proteins in combination with at least one cannabinoid compound are present in skin cells and the cells are exposed to oxidants or UV light, the CLIC proteins in combination with the cannabinoid compound may protect the skin cells from oxidative stress and/or UV damage.

The transdermal formulation described herein may comprise one or more CLIC proteins and one or more cannabinoid compounds in a therapeutically and/or prophylactically effective amount, and a carrier.

In some embodiments, the transdermal formulation may comprise or consist of one or more CLIC proteins, one or more cannabinoid compounds, a carrier, optionally one or more additives, optionally a penetration enhancer, and optionally a buffer.

The CLIC protein may be selected from the group comprising or consisting of CLIC1, CLIC2, CLIC3, CLIC4, CLIC5, CLIC6, or combinations thereof. In one embodiment, the CLIC protein may be selected from CLIC1, CLIC4, or a combination of both. In some embodiments, the CLIC proteins may be recombinant CLIC proteins. In some embodiments, the transdermal formulation may comprise or consist of one or more CLIC proteins and one or more cannabinoid compounds, wherein the CLIC protein is selected from CLIC1, CLIC4, or both, the cannabinoid compound is selected from cannabidiol (CBD), a carrier, optionally one or more additives, optionally a penetration enhancer, and optionally a buffer. For example, the transdermal formulation may comprise or consist of CLIC1, CLIC4, cannabidiol (CBD), a carrier, optionally one or more additives, optionally a penetration enhancer, and optionally a buffer. One or more advantages of the present disclosure, according to at least some embodiments or examples as described herein, is that a transdermal formulation can be prepared from one or more CLIC proteins comprising or consisting of CLIC1 and/or CLIC4 and one or more cannabinoid compounds comprising or consisting of cannabidiol (CBD).

In some embodiments, two different CLIC proteins may be combined. When two CLIC proteins are combined, the ratio of the CLIC proteins may be selected from 100:0, 90:10, 80:20, 70:30, 60:40, 50:50, 40:60, 30:70, 20:80, 10:90 or 0:100. The ratio of the CLIC proteins can be in a range provided by any two of these values. For example the ratio of CLIC1:CLIC4 may be selected from 100:0, 90:10, 80:20, 70:30, 60:40, 50:50, 40:60, 30:70, 20:80, 10:90 or 0:100. The ratio of CLIC1:CLIC4 can be in a range provided by any two of these values. In some embodiments, the ratio of CLIC1:CLIC4 is from 10:90 to 60:40. In some embodiments, the ratio of CLIC1:CLIC4 is 10:90. In some embodiments, the ratio of CLIC1:CLIC4 is 60:40.

It will be appreciated that CLIC1 is a member of a family of six human proteins called the Chloride Intracellular Channel Proteins (CLICs). For the purposes of nomenclature only and not limitation, exemplary sequences of human CLIC1, CLIC2, CLIC3, CLIC4, CLIC5, and CLIC6 are set out in Swiss-Prot Accession 000299, 015247, 095833, Q9Y696, Q9NZA1 and Q96NY7 respectively. Numerous published studies have placed the CLIC proteins within the superfamily of the Glutathione S-transferase (GST) fold proteins, and with particular structural similarity to the GST omega-class, as well as similarities to plant dehydroascorbate reductases (DHAR). The CLIC family members are highly conserved in vertebrates, with the high degree of conservation between these six vertebrate paralogues suggesting that they evolved from duplication of a single ancestral protein. Furthermore, structural studies have also shown that the six CLICs are related to the thioredoxin fold family (which are oxidoreductase enzymes). Oxidoreductase enzymes structurally have a catalytic site that includes a pair of cysteine residues. This catalytic site, has the ability to mediate the oxidation-reduction of specific target substrates. Glutaredoxin proteins structurally belong to the thioredoxin superfamily of enzymes. These proteins are classified into monothiol Grxs, which contains only one cysteine residue in their active site motif (C-X-X-S), or dithiol Grxs, which have two cysteine residues in their catalytic active site motif (C-X-X-C). Importantly, all six CLIC proteins contain a Glutaredoxin-like active site comprised of an active Cysteine residue (located at position Cys24 in CLIC1). Is it to be understood that CLIC 1, 4, 5, 6 are monothiol, while CLICs 2 and 3 contain a dithiol motif. The CLICs are structurally similar to GST omega 1, which contains a unique active cysteine residues that promote the formation of a disulfide bridge with glutathione. The enzymatic activity of the mammalian GST-omega class is distinct to other GST classifications and also resembles the glutaredoxin proteins, along with similar structure to small redox thioredoxin proteins. It also has the ability to maintain a healthy redox via a deglutathionylation reaction which contributes to decreasing disulfide linkages within the cellular environment. This detoxifying reaction occurs within its monothiol GSH binding sites (or G-site), that contains a single cysteine residue (Cys-Gly-Phe-Ser) and exists within the conserved thioredoxin domain. It will be appreciated that the CLIC proteins contain an enzyme site motif highly homologous to the G-site motif found in the Glutaredoxins and GST omega protein and may exhibit oxidoreductase, antioxidant, cellular protective activity, and may therefore provide one or more advantages of the present disclosure. Based on the above, it will be appreciated that CLIC1, CLIC2, CLIC3, CLIC4, CLIC5, and CLIC6, or, for example, CLIC1, CLIC3, and CLIC4, each share a structural link, and therefore expected to exhibit oxidoreductase, antioxidant, cellular protective activity, and may therefore provide one or more advantages of the present disclosure.

The transdermal formulations described herein may comprise one or more CLIC proteins, by weight % of the formulation, in an amount of at least about 0.0001, 0.0005, 0.001, 0.005, 0.01, 0.05, 0.1, 0.5, 1, 2, 5, 10, 15, 20, 25, 30, 35, or 40. The formulations described herein may comprise one or more CLIC proteins, by weight %, in an amount less than about 40, 35, 30, 25, 20, 15, 10, 5, 2, 1, 0.5, 0.1, 0.05, 0.0, 0.005, 0.001, 0.0005 or 0.0001. The formulations described herein may comprise one or more CLIC proteins, by weight %, in an amount in a range provided by any two of these upper and/or lower values, for example between about 0.0001 and 10 wt %, about 0.0005 and 5 wt %, or about 0.001 and 2 wt %, based on total weight of the formulation.

Generally, the transdermal formulation comprises one or more CLIC proteins and one or more cannabinoid compounds in an amount that is a therapeutically or prophylactically effective amount. In some embodiments, the therapeutically or prophylactically effective amount is provided by a single dose or applications. In some embodiments, the therapeutically effective amount is provided by one or more doses or applications administered as part of a course of treatment, for example, 1, 2, 3, 4, 5, 6, 7, 8, 9, 10, 11, 12, 13, 14, 15, 16, 17, 18, 19, 20, 21, 22, 23, 24, 25, 26, 27 or greater than 27 doses or applications.

The person skilled in the art would understand that the amount of one or more CLIC proteins and the one or more cannabinoid compounds present in the formulation will vary depending on the other ingredients present in the formulation, the desired effect and the like. In some embodiments, the formulation comprises one or more CLIC proteins in an amount between about 0.01 to 1200 mg, 0.05 to 500 mg, 0.1 to 50 mg. In some embodiments the weight ratio of the one or more CLIC proteins to carrier may be about 1:1, 1:4, 1:5, 1:10, 1:20, 1:25, 1:50, 1:100, 1:1000, 1:10000 or 1:100000. In some embodiments, the formulation comprises one or more cannabinoid compounds in an amount between about 0.0001 to 1200 mg/ml. In some embodiments the weight ratio of the one or more cannabinoid compounds to carrier may be about 1:1, 1:4, 1:5, 1:10, 1:20, 1:25, 1:50, 1:100, 1:1000, 1:10000 or 1:100000.

It will be appreciated that the term “cannabinoid compound” refers compounds found in the cannabis plant or synthetic compounds that can interact with the endocannabinoid system. As used herein, the term “cannabinoid compound” includes lipophilic compounds found in cannabis, and includes classical and non-classical cannabinoids. Classical cannabinoids are structurally related to THC. Non-classical cannabinoids (cannabimimetics) include aminoalkylindoles, 1,5-diarylpyrazoles, quinolines, and arylsulfonamindes as well as eicosanoids relates to endocannabinoids. In some embodiments, the one or more cannabinoid compounds may be selected from the group consisting of cannabidiol (CBD), cannabidiol A (CBDA) cannabigerol (CBG), cannabichromene (CBC), cannabicyclol (CBL), cannabinol (CBN), cannabigerolic acid (CBGA), cannabidivarin (CBDV), cannabichromevarin (CBCV), cannabigerovarin (CBGV), cannabigerol monomethyl ether (CBGM), or a combination thereof. In one example, the cannabinoid compound may be cannabidiol (CBD). In some embodiments, the cannabinoid compound may be present in the form of a cannabinoid extract or isolate having less than 1, 0.5, or 0.3 wt % of any of tetrahydrocannabinol (THC), tetrahydrocannabivarin (THCV) and tetrahydrocannabinolic acid (THCA). The formulation may comprise the one or more cannabinoid compounds in a concentration between about 0.0001 mg/ml to about 1200 mg/ml.

It will be appreciated that the transdermal formulation may be provided in various forms depending on the application and subject. For example, the formulation may be in the form of an aqueous solution, alcohol solution, ointment, cream, lotion, gel, paste, salve, spray, controlled-release matrix, transdermal delivery system, patch formulation, or any combination thereof.

### Carrier

The transdermal formulation comprises a carrier. The carrier may be an aqueous carrier. The carrier may comprise or consist of water, one or more alcohols, one or more lanolin alcohols, one or more fatty alcohols, one or more glycols, one or more oils, one or more waxes, one or more phospholipids, one or more sterols, isopropyl myristate, oleic acid, or combinations thereof. For example alcohols may include, but are not limited to, ethanol, isopropyl alcohol, benzyl alcohol. For example, fatty acids may include, but are not limited to, cetearyl alcohol, cetyl alcohol. For example, glycols may include, but are not limited to, propylene glycol, polypropylene glycols. For example, oils and waxes may include, but are not limited to, mineral oils, paraffins.

The carrier may be in an amount between about 2% and about 99.99% based on total weight of the formulation. The carrier can be provided in an amount (as a weight % based on total weight of the formulation) of less than about 99.99, 99.95, 99.9, 99.5, 99, 98, 95, 90, 85, 80, 75, 70, 65, 60, 55, 50, 45, 40, 35, 30, 25, 20, 15, 10, 5, or 2. The carrier can be provided in an amount (as a weight % based on total weight of the formulation) of at least about 2, 5, 10, 15, 20, 25, 30, 35, 40, 45, 50, 55, 60, 65, 70, 75, 80, 85, 90, 95, 98, 99, 99.5, 99.9, 99.95 or 99.99. The carrier can be in a range provided by any two of these upper and/or lower amounts.

The carrier may be suitably one or more substances which may also act as diluents, penetration enhancers, viscosity enhancers, or film formers. For example, the solvent carrier may be provided as a penetration enhancing compound. The carrier material may also include encapsulating material, for example, that may assist with liposomal or lipid particle preparation.

One or more advantages of the present disclosure, according to at least some embodiments or examples as described herein, is that a carrier present in the formulation may not only provide stability for the CLIC protein and the cannabinoid compound to maintain its activity but also allow transport/penetration of the CLIC protein and the cannabinoid compound through dermal layers to enable the CLIC proteins and the cannabinoid compounds unexpected protective and/or therapeutic effects.

### Optional Additive

In some embodiments the transdermal formulation can comprise or consist of one or more additives. The one or more additives may be selected from an antioxidant, a preservative, a stabilizer, a binder, a compaction agent, a lubricant, a dispersion enhancer and/or a colouring agent. For example, the one or more additives may be selected from antioxidants, preservatives, salts (e.g. NaCl), sugars (e.g. sucrose and trehalose), sugar alcohols (e.g. mannitol and sorbitol), polysaccharides, vitamins, essential oils, rheology modifiers, transdermal compounding bases or gels, lipoic acid, xanthan gums, and surfactants (e.g. polysorbate 80 (Tween 80) and polysorbate 20 (Tween 20)).

The transdermal formulation may comprise a nutrient. In some embodiments the formulation may comprise at least one carbohydrate. A “carbohydrate” refers to a sugar or polymer of sugars. The terms “saccharide,” “polysaccharide,” “carbohydrate,” and “oligosaccharide” may be used interchangeably. Most carbohydrates are aldehydes or ketones with many hydroxyl groups, usually one on each carbon atom of the molecule. Carbohydrates generally have the molecular formula CₙH₂ₙOₙ. A carbohydrate can be a monosaccharide, a disaccharide, trisaccharide, oligosaccharide, or polysaccharide. The most basic carbohydrate is a monosaccharide, and may include glucose, galactose, mannose, ribose, arabinose, xylose, and fructose. Disaccharides are two joined monosaccharides. Examples of disaccharides may include sucrose, maltose, cellobiose, trehalose, and lactose. Typically, an oligosaccharide includes between three and six monosaccharide units (e.g., raffinose, stachyose), and polysaccharides include six or more monosaccharide units. Examples of polysaccharides may include starch, glycogen, and cellulose. Carbohydrates can contain modified saccharide units such as 2′-deoxyribose wherein a hydroxyl group is removed, 2′-fluororibose wherein a hydroxyl group is replace with a fluorine, or N-acetylglucosamine, a nitrogen-containing form of glucose (e.g., 2′-fluororibose, deoxyribose, and hexose). Carbohydrates can exist in many different forms, for example, conformers, cyclic forms, acyclic forms, stereoisomers, tautomers, anomers, and isomers. For example, the polysaccharide may be selected from the group comprising or consisting of dextran sulfate, pectin, modified pectin, insoluble 1,3-β-D glucan, micronized 1,3-β-D glucan, soluble 1,3-β-D glucan, phosphorylated 1,3-β-D glucan, aminated 1,3-β-D glucan and carboxymethylated 1,3-β-D glucan, sulfated 1,3-β-D glucan, insoluble 1,3/1,6-β-D glucan, micronized 1,3/1,6-β-D glucan, soluble 1,3/1,6-β-D glucan, phosphorylated 1,3/1,6-β-D glucan, aminated 1,3/1,6-β-D glucan and carboxymethylated 1,3/1,6-β-D glucan or sulfated 1,3/1,6-β-D glucan.

In some embodiments the transdermal formulation may comprise at least one lipid. A “lipid” may comprise fats, oils, triglycerides, cholesterol, phospholipids, fatty acids in any form including free fatty acids. Fats, oils and fatty acids can be saturated, unsaturated (cis or trans) or partially unsaturated (cis or trans). In some embodiments the lipid comprises at least one fatty acid selected from lauric acid (12:0), myristic acid (14:0), palmitic acid (16:0), palmitoleic acid (16:1), margaric acid (17:0), heptadecenoic acid (17:1), stearic acid (18:0), oleic acid (18:1), linoleic acid (18:2), linolenic acid (18:3), octadecatetraenoic acid (18:4), arachidic acid (20:0), eicosenoic acid (20:1), eicosadienoic acid (20:2), eicosatetraenoic acid (20:4), eicosapentaenoic acid (20:5) (EPA), docosanoic acid (22:0), docosenoic acid (22:1), docosapentaenoic acid (22:5), docosahexaenoic acid (22:6) (DHA), and tetracosanoic acid (24:0).

In some embodiments the transdermal formulation may comprise at least one supplemental mineral or mineral source. Examples of minerals include, without limitation: chloride, sodium, calcium, iron, chromium, copper, iodine, zinc, magnesium, manganese, molybdenum, phosphorus, potassium, and selenium. Suitable forms of any of the foregoing minerals include soluble mineral salts, slightly soluble mineral salts, insoluble mineral salts, chelated minerals, mineral complexes, non-reactive minerals such as carbonyl minerals, and reduced minerals, and combinations thereof.

In additional embodiments the transdermal formulation may comprise at least one supplemental vitamin. The at least one vitamin may be fat-soluble or water soluble vitamins. Suitable vitamins include but are not limited to vitamin C, vitamin A, vitamin E, vitamin B12, vitamin K, riboflavin, niacin, vitamin D, vitamin B6, folic acid, pyridoxine, thiamine, pantothenic acid, and biotin. Suitable forms of any of the foregoing are salts of the vitamin, derivatives of the vitamin, compounds having the same or similar activity of the vitamin, and metabolites of the vitamin. For example, the one or more vitamins may be selected from the group comprising or consisting of cholecalciferol, α-tocopherol, β-tocopherol, 8-tocopherol, γ-tocopherol, α-tocotrienol, β-tocotrienol, δ-tocotrienol, γ-tocotrienol, and combinations thereof.

Various other additives may be included in the transdermal formulation. These include, but are not limited to, antioxidants, astringents, perfumes, preservatives, vitamins, emollients, pigments, dyes, humectants, propellants, and sunscreen agents, as well as other classes of materials whose presence may be pharmaceutically or otherwise desirable. Other non-limiting examples of optional additives are as follows: preservatives such as sorbate; solvents such as isopropanol and propylene glycol; astringents such as menthol and ethanol; emollients such as polyalkylene methyl glucosides; humectants such as glycerine; emulsifiers such as glycerol stearate, PEG-100 stearate, polyglyceryl-3 hydroxylauryl ether, and polysorbate 60; sorbitol and other polyhydroxyalcohols such as polyethylene glycol; sunscreen agents such as octyl methoxyl cinnamate and butyl methoxy benzoylmethane; antioxidants such as ascorbic acid (vitamin C), α-tocopherol (Vitamin E), β-tocopherol, γ-tocopherol, δ-tocopherol, ε-tocopherol, ζ-tocopherol, θ-tocopherol, η-tocopherol, and retinol (vitamin A); essential oils, ceramides, essential fatty acids, mineral oils, vegetable oils (e.g., soya bean oil, palm oil, liquid fraction of shea butter, sunflower oil), animal oils (e.g., perhydrosqualene), synthetic oils, silicone oils or waxes (e.g., cyclomethicone and dimethicone), fluorinated oils (generally perfluoropolyethers), fatty alcohols (e.g., cetyl alcohol), and waxes (e.g., beeswax, carnauba wax, and paraffin wax); skin-feel modifiers; and thickeners and structurants such as swelling clays and cross-linked carboxypolyalkylenes. For example, the preservative may be methylparaben, methylparaben sodium, propylparaben, propylparaben sodium, potassium sorbate benzalkonium chloride, benzthonium chloride or any combination thereof. For example, the antioxidant may be ascorbic acid, sodium ascorbate, sodium bisulfite, sodium metabisulfate, curcumin, curcumin derivatives, ursolic acid, resveratrol, resveratrol derivatives, alpha-lipoic acid, monothioglycerol, α-tocopherol, β-tocopherol, γ-tocopherol, δ-tocopherol, ε-tocopherol, ζ-tocopherol, θ-tocopherol, η-tocopherol, retinol, or any combination thereof. For example, the one or more vitamins may be selected from the group comprising of cholecalciferol, α-tocopherol, β-tocopherol, δ-tocopherol, γ-tocopherol, α-tocotrienol, β-tocotrienol, δ-tocotrienol, γ-tocotrienol, and combinations thereof. For example, the one or more essential oils may be selected from the group comprising rosemary essential oil, birch essential oil, balsam fir essential oil, peppermint essential oil, marjoram essential oil, helichrysum essential oil, thyme essential oil, frankincense essential, clove essential oil, turmeric essential oil, orange essential oil, or any combination thereof.

Optionally, the transdermal formulation may comprise a viscosity enhancer, and/or film former. A viscosity enhancer increases the viscosity of the formulation so as to inhibit its spread beyond the site of application.

A film former, when it dries, forms a protective film over the site of application. A film former may act additionally as a carrier. Solutions that dry to form a film are sometimes referred to as paints.

Other additives may include materials that condition the skin (particularly, the upper layers of the skin in the stratum corneum) and keep it soft by retarding the decrease of its water content and/or protect the skin. For example, such conditioners and moisturizing agents include pyrrolidine carboxylic acid and amino acids; organic antimicrobial agents such as 2,4,4′-trichloro-2-hydroxy diphenyl ether (triclosan) and benzoic acid. Further additives include anti-inflammatory agents such as acetylsalicylic acid and glycyrrhetinic acid; anti-seborrhoeic agents such as retinoic acid; vasodilators such as nicotinic acid; inhibitors of melanogenesis such as kojic acid; and mixtures thereof.

In other embodiments, the transdermal formulation can include alpha hydroxyacids, alpha ketoacids, polymeric hydroxyacids, moisturizers, collagen, marine extract, and antioxidants such as ascorbic acid (vitamin C) and/or α-tocopherol (Vitamin E). Sunscreens may also be included. Additional, components such as enzymes, herbs, plant extracts, glandular or animal extracts can be added to the formulation.

The transdermal formulation may also comprise antimicrobial agents, to prevent spoilage upon storage, i.e., to inhibit growth of microbes such as yeasts and moulds. Suitable antimicrobial agents are typically selected from the group comprising or consisting of the methyl and propyl esters of p-hydroxybenzoic acid (i.e., methyl and propyl paraben), sodium benzoate, sorbic acid, imidurea, and combinations thereof.

The transdermal formulation may also comprise irritation-mitigating additives to minimize or eliminate the possibility of skin irritation or skin damage resulting from the chemical entity to be administered, or other components of the composition. Suitable irritation-mitigating additives include, for example: α-tocopherol; monoamine oxidase inhibitors, particularly phenyl alcohols such as 2-phenyl-1-ethanol; glycerin; salicylates; ascorbates; ionophores such as monensin; amphiphilic amines; ammonium chloride; N-acetylcysteine; capsaicin; and chloroquine. The irritation-mitigating additive, if present, can be incorporated into the transdermal formulation at a concentration effective to mitigate irritation or skin damage, typically representing not more than about 15 wt. %, more typically not more than about 5 wt. %, of the formulation.

The amounts of these various additives are those conventionally used in the skin care field. In some embodiments, the additive may be present in the formulation and may be provided in an amount from about 0.01 to about 15 wt. % based on total weight of the transdermal formulation. The additive can be provided in an amount (as a weight % based on total weight of the transdermal formulation) of less than about 15, 10, 5, 1, 0.5, 0.1, 0.05, or 0.01. The additive can be provided in an amount (as a weight % based on total weight of the transdermal formulation) of at least about 0.01, 0.05, 0.1, 0.5, 1, 5, 10, or 15. The additive can be in a range provided by any two of these upper and/or lower amounts.

### Optional Penetration Enhancer

In some embodiments, the transdermal formulation may comprise or consist of a penetration enhancer. For example, a penetration enhancing compound may be incorporated in the transdermal formulation to facilitate the skin penetration of one or more CLIC proteins and the one or more cannabinoid compounds. In an embodiment, the penetration enhancer may include water, sulphoxides (e.g. dimethylsulphoxide, DMSO), azones (e.g. laurocapram), pyrrolidones (e.g. 2-pyrrolidone), alcohols and alkanols (e.g. ethanol, or decanol), glycols (e.g. propylene glycol, a common excipient in topically applied dosage forms), surfactants (also common in dosage forms), fatty acids, and terpenes and terpenoids. It will be appreciated that the carrier, as described herein, may also have penetration enhancing properties.

In some embodiments, the penetration enhancer may be present in the transdermal formulation and may be provided in an amount from about 0.01 to about 15 wt. % based on total weight of the transdermal formulation. The penetration enhancer can be provided in an amount (as a weight % based on total weight of the transdermal formulation) of less than about 15, 10, 5, 1, 0.5, 0.1, 0.05, or 0.01. The penetration enhancer can be provided in an amount (as a weight % based on total weight of the transdermal formulation) of at least about 0.01, 0.05, 0.1, 0.5, 1, 5, 10, or 15. The penetration enhancer can be in a range provided by any two of these upper and/or lower amounts.

### Optional Buffer

In some embodiments, the transdermal formulation may comprise or consist of a buffer. For example, the buffer may be selected from the group comprising glycerol, glutathione, phosphate-based buffer (e.g. sodium and potassium hydrogen phosphate buffers), acetate buffer, phosphate buffer, citrate buffer, TRIS buffer, maleate buffer, succinate buffer, histidine buffer, amino acids (e.g. histidine, arginine, and glycine), or combinations thereof. Other non-limiting examples of suitable buffering agents may include sodium citrate, magnesium carbonate, magnesium bicarbonate, calcium carbonate, and calcium bicarbonate.

A pH buffering agent(s) can, if employed and when dissolved in an aqueous component of the composition, provide a pH in the range of 5 to 7. For example, the pH may be about pH 5.5.

In some embodiments, the buffer may be present in the transdermal formulation and may be provided in an amount from about 0.01 to about 15 wt. % based on total weight of the transdermal formulation. The buffer can be provided in an amount (as a weight % based on total weight of the transdermal formulation) of less than about 15, 10, 5, 1, 0.5, 0.1, 0.05, or 0.01. The buffer can be provided in an amount (as a weight % based on total weight of the transdermal formulation) of at least about 0.01, 0.05, 0.1, 0.5, 1, 5, 10, or 15. The buffer can be in a range provided by any two of these upper and/or lower amounts.

### Transdermal Formulation Forms

In some embodiments, the formulation may be in the form of an aqueous solution, alcohol solution, ointment, cream, lotion, gel, paste, salve, spray, controlled-release matrix, transdermal delivery system, patch formulation, or any combination thereof.

Cream bases are water-washable, and may comprise an oil phase, an emulsifier, and an aqueous phase. The oil phase, also called the “internal” phase, may comprise petrolatum and a fatty alcohol such as cetyl or stearyl alcohol. The aqueous phase usually, although not necessarily, exceeds the oil phase in volume, and may comprise a humectant. The emulsifier in a cream formulation may comprise a nonionic, anionic, cationic or amphoteric surfactant.

Lotions, are preparations to be applied to the skin surface without friction, and are typically liquid or semiliquid preparations in which one or more CLIC proteins are present in a water or alcohol base. Lotions are usually suspensions of solids, and preferably, comprise a liquid oily emulsion of the oil-in-water type. Lotions can be used for treating large body areas, because of the ease of applying a more fluid composition. It is generally necessary that the insoluble matter in a lotion be finely divided.

Lotions typically contain suspending agents to produce better dispersions as well as compounds useful for localizing and holding the one or more CLIC proteins and one or more cannabinoid compounds (e.g. cannabidiol (CBD) in contact with the skin, e.g., methylcellulose, sodium carboxymethyl-cellulose, or the like.

Solutions are homogeneous mixtures and may be prepared by dissolving one or more CLIC proteins and one or more cannabinoid compounds in a carrier (e.g. aqueous soluble solvent carrier) such that the molecules of the dissolved CLIC proteins and cannabinoid compounds are dispersed among those of the carrier. The solution may contain other pharmaceutically or cosmetically acceptable chemicals to buffer, stabilize or preserve the one or more CLIC proteins and/or one or more cannabinoid compounds. Examples of solvent carriers that may be used in preparing topical solutions are water, alcohols (e.g. ethanol, isopropyl alcohol, benzyl alcohol), lanolin alcohols, fatty alcohols (e.g. cetearyl alcohol, cetyl alcohol), glycols (e.g. propylene glycol, polypropylene glycols), oils and waxes (e.g. mineral oils, paraffins), isopropyl myristate, or oleic acid. These can be applied in any manner, such as spraying them on the skin, or painting them on the skin.

Gels, hydrogels are semisolid, suspension-type systems. Single-phase gels contain organic macromolecules distributed substantially uniformly throughout the carrier liquid, which is typically aqueous, but also, preferably, contain an alcohol, and, optionally, an oil. Some “organic macromolecules,” of use, specifically gelling agents, are crosslinked acrylic acid polymers such as the “carbomer” family of polymers, e.g., carboxypolyalkylenes that are commercially available as Carbopol®. Also of use are hydrophilic polymers such as polyethylene oxides, polyoxyethylene-polyoxypropylene copolymers and polyvinylalcohol; cellulosic polymers such as hydroxypropyl cellulose, hydroxyethyl cellulose, hydroxypropyl methylcellulose, hydroxypropyl methylcellulose phthalate, and methyl cellulose; gums such as tragacanth and xanthan gum; sodium alginate; and gelatin. In order to prepare a uniform gel, dispersing agents such as alcohol or glycerin can be added, or the gelling agent can be dispersed by trituration, mechanical mixing or stirring, or combinations thereof. These gels are of use in the methods disclosed herein.

Ointments can also be used in the disclosed methods. Ointments are semisolid preparations that are typically based on petrolatum or other petroleum derivatives. The specific ointment base to be used, as will be appreciated by those skilled in the art, is one that will provide for a number of desirable characteristics, e.g., emolliency or the like. An ointment base is generally inert, stable, nonirritating, and nonsensitizing. Ointment bases are grouped in four classes: oleaginous bases; emulsifiable bases; emulsion bases; and water-soluble bases (see Remington: The Science and Practice of Pharmacy, 19th Ed. (Easton, PA: Mack Publishing Co., 1995), at pages 1399-1404). Oleaginous ointment bases include, for example, vegetable oils, fats obtained from animals, and semisolid hydrocarbons obtained from petroleum. Emulsifiable ointment bases, also known as absorbent ointment bases, contain little or no water and include, for example, hydroxystearin sulfate, anhydrous lanolin, and hydrophilic petrolatum. Emulsion or microemulsion ointment bases are either water-in-oil (W/O) emulsions, biocontinuous, or oil-in-water (O/W) emulsions, and include, for example, acetyl alcohol, glyceryl monostearate, lanolin, and stearic acid. Water-soluble ointment bases are prepared from polyethylene glycols of varying molecular weight.

Pastes are semisolid dosage forms in which the one or more CLIC proteins and one or more cannabinoid compounds may be suspended in a suitable base, and are also of use. Depending on the nature of the base, pastes are divided between fatty pastes or those made from single-phase aqueous gels. The base in a fatty paste is generally petrolatum or hydrophilic petrolatum or the like. The pastes made from single-phase aqueous gels generally incorporate carboxymethylcellulose or the like as a base.

It will be appreciated that the transdermal formulation described herein can be any form suitable for application to the body surface (e.g. applied to the skin of a subject). For example, the transdermal formulation may be in the form of a cream, lotion, sprays, solution, gel, ointment, paste, plaster, paint, bioadhesive, bandage, sprays, suspensions or the like, and/or may be prepared so as to contain liposomes, micelles, and/or microspheres. A transdermal formulation can be used in combination with an occlusive overlayer so that moisture evaporating from the body surface is maintained within the formulation upon application to the body surface and thereafter.

A cream, lotion, gel, ointment, paste or the like may be spread on the surface of the skin of a subject. A solution may be applied in the same way, but more typically will be applied with a dropper, swab, sprayer or the like, and carefully applied to the subject (e.g. to affected areas or areas in need thereof). The transdermal formulation can be applied directly to the target location, for example in a topical preparation such as an ointment, or as a part of a dressing or a bandage, or a patch. The formulation can be formulated as a unit dosage, for administration by any device for administration to the skin. The unit dosage may be a reservoir of the one or more CLIC proteins and the one or more cannabinoid compounds in a carrier, for example an adhesive carrier capable of adhering to the skin for a desired period of time such as at least a day or more.

In some embodiments, liquid dosage form preparations may include solutions, suspensions, and emulsions. It will be appreciated that aqueous solutions, suspensions, and emulsions, are prepared by dissolving or dispersing the formulation in water or other suitable liquid (such as an alcohol) and adding suitable optional additives, optional penetration enhancer and optional buffer.

Furthermore, it will be appreciated that the choice of carriers and/or additives will, at least in part, be dependent upon the dosage form.

### Processes for Preparing a Transdermal Formulation

The formulations may conveniently be presented in unit dosage form such as creams, ointments, gels, lotions, sprays and sterile aqueous solutions or suspensions and may be prepared by any of the methods well known in the art. All methods include the step of bringing one or more CLIC proteins and one or more cannabinoid compounds (e.g. cannabidiol (CBD)) into association with a carrier, one or more optional additives, an optional penetration enhancer, and an optional buffer.

In general, the formulations are prepared by bringing one or more CLIC proteins and one or more cannabinoid compounds (e.g. cannabidiol (CBD)) into association with a carrier to form a solution or a suspension. The one or more cannabinoid compounds may be provided in any suitable form. For example, the one or more cannabinoid compounds may be provided in the form of an aqueous solution, which may be prepared by dissolving the one or more cannabinoid compounds in water. The aqueous solution may comprise one or more additional components to improve the solubility of the one or more cannabinoid compounds in water, for example cyclodextrin as described in patent application WO 2007/093177, the entire contents of which are herein incorporated by reference. In particular, WO 2007/093177 page 5 line 1 to page 6 line 11; page 7 lines 23 to page 8 line 26; page 10 line 11 to page 15 line 6; page 21 line 1 to page 28 line 34; and page 30 line 28 to page 31 line 2. Accordingly, in some embodiments of the transdermal formulation described herein, the transdermal formulation comprises cyclodextrin.

For application to the skin, a therapeutically or prophylactically effective amount of the transdermal formulation can be locally administered to the skin. The transdermal formulation disclosed herein facilitate the penetration of one or more CLIC proteins and one or more cannabinoid compounds (e.g. cannabidiol (CBD)) through the epidermis. Such a formulation can be suitable for delivery of the CLIC protein(s) and cannabinoid compound(s) (e.g. cannabidiol (CBD)) to any suitable subject, such as but not limited to, a human subject, and can be manufactured in a manner that is itself known, for example, by means of conventional mixing, dissolving, granulating, emulsifying, encapsulating (e.g. micelle or liposome formulations), entrapping or lyophilizing processes.

In some embodiments, the transdermal formulation may in the form of an ointment, cream, lotion, emulsion, gel or vesicular delivery systems. Such formulations may include agents that promote penetration of the CLIC proteins and cannabinoid compound(s) (e.g. cannabidiol (CBD)) through the epidermis (e.g. a penetration enhancer). Various other additives known in the art may be included in the formulations as described herein.

In some embodiments, the process for preparing a transdermal formulation comprising an effective amount of one or more CLIC proteins, one or more cannabinoid compounds (e.g. cannabidiol (CBD)) and a carrier comprising the steps of: providing a solution comprising one or more CLIC proteins, one or more cannabinoid compounds (e.g. cannabidiol (CBD)) and a carrier; and adding to the solution one or more optional additives, an optional penetration enhancer, and an optional buffer.

In some embodiments, the transdermal formulation may comprise additional inactive materials in order to produce a final product, which may be in single dosage unit or in a multi-dose format. The preparation of the liquid form may be an emulsion or a microemulsion. The preparation of the topical form may be encapsulation (e.g. by formation of lipid-based particles such as liposomes) or microencapsulation (e.g. by formation of micelles). In some embodiments, the transdermal formulation can be formulated as a micelle formulation or a liposome formulation. The micelles and liposomes may be formed using various known methodologies, such as thin-film hydration method, microemulsification, sonication, membrane extrusion, freeze-thaw method, ether injection method, ethanol injection method, reverse phase evaporation method, dehydration-rehydration, and calcium-induced fusion method.

Typically, the micelle and liposome formations may be verified by laser light obscuration analysis and Dynamic Light Scattering (DLS). In some embodiments, the present disclosure provides the formation of a micelle formulation, wherein the micelle formulation comprises one or more CLIC proteins in combination with one or more cannabinoid compounds (e.g. cannabidiol (CBD)), as well as a method for the manufacture of a micelle formulation comprising one or more CLIC proteins in combination with one or more cannabinoid compounds (e.g. cannabidiol (CBD)). For example, the micelle formulation may comprise a CLIC protein and a cannabinoid compound, wherein the CLIC protein may be CLIC1 and the cannabinoid compound may be cannabidiol (CBD). In another example, the micelle formulation may comprise a CLIC protein and a cannabinoid compound, wherein the CLIC protein may be CLIC4 and the cannabinoid compound may be cannabidiol (CBD). In some embodiments, the micelle formulation may consist of one or more CLIC proteins and one or more cannabinoid compounds, wherein the CLIC protein is selected from CLIC1, CLIC4, or both, and wherein the cannabinoid compound is cannabidiol (CBD), a carrier, optionally one or more additives, optionally a penetration enhancer, and optionally a buffer. To obtain the desired final formulation, the suspension of micelles is mixed to produce a homogenous micelle formulation of one or more CLIC proteins in combination with one or more cannabinoid compounds.

In some embodiments, the present disclosure provides the formation of a liposome formulation, wherein the liposome formulation comprises one or more CLIC proteins and one or more cannabinoid compounds, as well as a method for the manufacture of a liposome formulation comprising one or more CLIC proteins and one or more cannabinoid compounds. For example, the liposome formulation may comprise a CLIC protein and a cannabinoid compound, wherein the CLIC protein may be CLIC1 and the cannabinoid compound may be cannabidiol (CBD). In another example, the liposome formulation may comprise a CLIC protein and a cannabinoid compound, wherein the CLIC protein may be CLIC4 and the cannabinoid compound may be cannabidiol (CBD). In some embodiments, the liposome formulation may consist of one or more CLIC proteins and one or more cannabinoid compounds, wherein the CLIC protein is selected from CLIC1, CLIC4, or both, and wherein the cannabinoid compound is cannabidiol (CBD), a carrier, optionally one or more additives, optionally a penetration enhancer, and optionally a buffer. In some embodiments, two different CLIC proteins may be combined. When two CLIC proteins are combined, the ratio of the CLIC proteins may be selected from 100:0, 90:10, 80:20, 70:30, 60:40, 50:50, 40:60, 30:70, 20:80, 10:90 or 0:100. The ratio of the CLIC proteins can be in a range provided by any two of these values. For example the ratio of CLIC1:CLIC4 may be selected from 100:0, 90:10, 80:20, 70:30, 60:40, 50:50, 40:60, 30:70, 20:80, 10:90 or 0:100. The ratio of CLIC1:CLIC4 can be in a range provided by any two of these values.

To obtain the desired final formulation, the suspension of liposome is mixed to produce a homogenous liposome formulation of one or more CLIC proteins in combination with one or more cannabinoid compounds (e.g. cannabidiol (CBD)).

The size of micelles in the micelle formulation may be in a range from about 2 nm to about 20 nm. In some embodiments, the size of the micelles in the micelle formulation may be at least about (nm) 2, 2.5, 3, 3.5, 4, 4.5, 5, 5.5, 6, 6.5, 7, 7.5, 8, 8.5, 9, 9.5, 10, 12, 14, 16, 17, 18, 19 or 20. In some embodiments, the size of the micelles in the micelle formulation may be less than about (nm) 20, 19, 18, 17, 16, 15, 14, 12, 10, 9, 8.5, 8, 7.5, 7, 6, 5.5, 5, 4.5, 4, 3.5, 3, 2.5, or 2. The size of the micelles in the micelle formulation can be in a range provided by any two of these upper and/or lower values.

The size of liposomes in the liposome formulation may be in a range from about 0.02 μm to about 300 μm. In some embodiments, the size of the liposomes in the liposome formulation may be at least about (μm) 0.02, 0.05, 0.1, 0.5, 1, 5, 10, 20, 40, 60, 80, 100, 120, 140, 160, 180, 200, 220, 240, 260, 280 or 300. In some embodiments, the size of the liposomes in the liposome formulation may be less than about (μm) 300, 280, 260, 240, 220, 200, 180, 160, 140, 120, 100, 80, 60, 40, 20, 10, 5, 1, 0.5, 0.1, 0.05 or 0.02. The size of the liposomes in the liposome formulation can be in a range provided by any two of these upper and/or lower values.

In some embodiments, the amount of the one or more CLIC proteins in the transdermal formulation is in a therapeutically effective amount. The amount of the one or more CLIC proteins may be in a range between about 0.0001 to about 10 wt % based on the total weight of the formulation. The one or more CLIC proteins can be provided in an amount (as a weight % based on total weight of the formulation) of less than about 40, 35, 30, 25, 20, 15, 10, 5, 2, 1, 0.5, 0.1, 0.05, 0.01, 0.005, 0.001, 0.0005 or 0.0001. The one or more CLIC proteins can be provided in an amount (as a weight % based on total weight of the formulation) of at least about 0.0001, 0.0005, 0.001, 0.005, 0.01, 0.05, 0.1, 0.5, 1, 2, 5, 10, 15, 20, 25, 30, 35, or 40. The one or more CLIC proteins can be in a range provided by any two of these upper and/or lower amounts. For example, amount of the one or more CLIC proteins may be in a range between about 0.0001 to about 10 wt %, about 0.0005 to about 5 wt %, or about 0.001 to about 2 wt %, based on the total weight of the formulation.

The concentration of the one or more CLIC proteins in the transdermal formulation may be in a range from about 0.001 mg/mL to about 5 mg/mL. In some embodiments, the concentration of the one or more CLIC proteins in the transdermal formulation may be in a range from about 0.005 mg/mL to about 2 mg/mL, from about 0.008 mg/mL to about 0.2 mg/mL, or from about 0.01 mg/mL to about 1 mg/mL. The concentration of the one or more CLIC proteins in the transdermal formulation may be at least about (mg/mL) 0.001, 0.002, 0.005, 0.008, 0.01, 0.02, 0.05, 0.08, 0.1, 0.2, 0.5, 1, 2 or 5. The concentration of the one or more CLIC proteins in the transdermal formulation may be less than about (mg/mL) 5, 2, 1, 0.5, 0.2, 0.1, 0.008, 0.005, 0.002, or 0.001. The concentration of the one or more CLIC proteins in the transdermal formulation can be in a range provided by any two of these upper and/or lower amounts.

In some embodiments, the ratio of CLIC protein to carrier in the transdermal formulation may be from about 1:1 to about 1:1000. The ratio of CLIC protein to carrier in the transdermal formulation may be at least about 1:1, 1:4, 1:5, 1:10, 1:20, 1:25, 1:50, 1:100, 1:1000, 1:10000 or 1:100000. The ratio of CLIC protein to carrier in the transdermal formulation may be in a range provided by any two of these upper and/or lower values.

In some embodiments, the concentration of the one or more cannabinoid compounds in the transdermal formulation may be in a range from about 0.0001 mg/ml to about 1200 mg/ml. The concentration of the one or more cannabinoid compounds in the transdermal formulation may be at least about (mg/mL) 0.0001, 0.0005, 0.001, 0.002, 0.005, 0.008, 0.01, 0.02, 0.05, 0.08, 0.1, 0.2, 0.5, 1, 2, 5, 10, 20, 50, 100, 200, 400, 600, 800, 1000, or 1200. The concentration of the one or more CLIC proteins in the transdermal formulation may be less than about (mg/mL) 1200, 1000, 800, 600, 400, 200, 100, 50, 20, 10, 5, 2, 1, 0.5, 0.2, 0.1, 0.008, 0.005, 0.002, 0.001, 0.0005, or 0.0001. The concentration of the one or more cannabinoid compound in the transdermal formulation can be in a range provided by any two of these upper and/or lower amounts.

In some embodiments, the ratio of cannabinoid compound to carrier in the transdermal formulation may be from about 1:1 to about 1:1000. The ratio of the one or more cannabinoid compound to carrier in the transdermal formulation may be at least about 1:1, 1:4, 1:5, 1:10, 1:20, 1:25, 1:50, 1:100, 1:1000, 1:10000 or 1:100000. The ratio of cannabinoid compound to carrier in the transdermal formulation may be in a range provided by any two of these upper and/or lower values.

In some embodiment, the amount of the one or more cannabinoid compounds in the transdermal formulation is in a therapeutically effective amount. The amount of the one or more cannabinoid compounds may be in a range between about 0.0001 to about 10 wt % based on the total weight of the formulation. The one or more cannabinoid compounds can be provided in an amount (as a weight % based on total weight of the formulation) of less than about 40, 35, 30, 25, 20, 15, 10, 5, 2, 1, 0.5, 0.1, 0.05, 0.01, 0.005, 0.001, 0.0005 or 0.0001. The one or more cannabinoid compounds can be provided in an amount (as a weight % based on total weight of the formulation) of at least about 0.0001, 0.0005, 0.001, 0.005, 0.01, 0.05, 0.1, 0.5, 1, 2, 5, 10, 15, 20, 25, 30, 35, or 40. The one or more cannabinoid compounds can be in a range provided by any two of these upper and/or lower amounts. For example, amount of the one or more cannabinoid compounds may be in a range between about 0.0001 to about 10 wt %, about 0.0005 to about 5 wt %, or about 0.001 to about 2 wt %, based on the total weight of the formulation.

The present disclosure may also provide a transdermal formulation prepared by the process described herein.

### Applications and Methods of Treatment

The present disclosure may provide for a method of reducing and/or preventing oxidative stress in cells of mammalian skin, the method comprising: topically administering a transdermal formulation to the mammalian skin, wherein the transdermal formulation comprises a therapeutically and/or prophylactically effective amount of one or more CLIC proteins, one or more cannabinoid compounds, and a carrier. The present disclosure may also provide for a use of a transdermal formulation, as described herein, for reducing and/or preventing oxidative stress in cells of mammalian skin. In some embodiments, reducing and/or preventing oxidative stress in cells of mammalian skin comprises increasing the viability of cells in mammalian skin compared to untreated cells, for example as measured by mammalian skin cell viability assays known in the art, including those described herein.

The present disclosure may provide for a method of rejuvenating UV damaged cells in mammalian skin, the method comprising: topically administering a transdermal formulation to the mammalian skin, wherein the transdermal formulation comprises a therapeutically effective amount of one or more CLIC proteins, one or more cannabinoid compounds, and a carrier. The present disclosure may also provide for a use of a transdermal formulation, as described herein, for rejuvenating UV damaged cells in mammalian skin. In some embodiments, rejuvenating UV damaged cells in mammalian skin comprises increasing the viability of cells in mammalian skin exposed to UV compared to untreated cells, for example as measured by mammalian skin cell viability assays known in the art, including those described herein.

The present disclosure may provide for a method of treating UV damaged cells and/or preventing UV damage in mammalian skin, the method comprising: topically administering a transdermal formulation to the mammalian skin, wherein the transdermal formulation comprises a therapeutically and/or prophylactically effective amount of one or more CLIC proteins, one or more cannabinoid compounds, and a carrier. The present disclosure may also provide for a use of a transdermal formulation, as described herein, for treating UV damaged cells and/or preventing UV damage in mammalian skin. In some embodiments, treating UV damaged cells and/or preventing UV damage in mammalian skin comprises increasing the viability of cells in mammalian skin exposed to UV compared to untreated cells, for example as measured by mammalian skin cell viability assays known in the art, including those described herein.

It will be appreciated that any one or more of the methods described herein may comprise steps of administering the formulation topically in the form of an ointment, lotion, cream, gel, salve, spray, controlled-release matrix, transdermal delivery system, patch formulation, or any combination thereof. The method may comprise steps of administering the formulation over a time period of about 1 day to about 6 months. The method may comprise steps of administering the formulation in a dosage of the one or more CLIC proteins in a range between about 5 mg to about 100 mg per day. The method may comprise steps of administering the formulation one, two, three or four times per day. It will be appreciated that the one or methods described herein wherein the mammalian skin, or cells therein, is that of a human patient in need of such treatment or prophylaxis.

Any subject, in need thereof, can be treated using the methods disclosed herein. The subject can be a human. In some embodiments, the subject is a child, such as a subject that is 17, 16, 15, 14, 13, 12, 11, 10, 9, 8, 7, 6, 5, 4, 3, 2 or 1 year(s) of age or less. The subject can be an infant, such as a subject of less than 1 year of age. In other embodiments, the subject is an adult, such as subject who is 18 years of age, greater than 20, 25, 30, 35, 40, 45, 50, 55, or 60 years of age. The subject can be a senior, such as a subject who is greater than 65, 70, 75, 80, 85, 90, or 95 years of age.

The present disclosure may also provide for a use of one or more CLIC proteins, which may be selected from the group consisting of: CLIC1, CLIC2, CLIC3, CLIC4, CLIC5, CLIC6, and combinations thereof, in combination with one or more cannabinoid compounds, in the manufacture of a transdermal medicament for the reduction of oxidative stress in cells of mammalian skin and/or for the prevention of oxidative stress to said cells of mammalian skin.

The present disclosure may also provide for a use of one or more CLIC proteins, which may be selected from the group consisting of: CLIC1, CLIC2, CLIC3, CLIC4, CLIC5, CLIC6, and combinations thereof, in combination with one or more cannabinoid compounds, in the manufacture of a transdermal medicament for the rejuvenation of UV damaged cells in mammalian skin.

The present disclosure may also provide for a use of one or more CLIC proteins, which may be selected from the group consisting of: CLIC1, CLIC2, CLIC3, CLIC4, CLIC5, CLIC6, and combinations thereof, in combination with one or more cannabinoid compounds, in the manufacture of a transdermal medicament for the treatment of UV damaged cells of mammalian skin and/or for the prevention of UV damage to said cells of mammalian skin.

The present disclosure may also provide for a transdermal formulation for use in reducing and/or preventing oxidative stress in cells of mammalian skin, the transdermal formulation comprising one or more CLIC proteins, which may be selected from the group consisting of CLIC1, CLIC2, CLIC3, CLIC4, CLIC5, CLIC6, and combinations thereof, in combination with one or more cannabinoid compounds. The use may comprise administering the transdermal formulation to the mammalian skin.

The present disclosure may also provide for a transdermal formulation for use in rejuvenating UV damaged cells in mammalian skin, the transdermal formulation comprising one or more CLIC proteins, which may be selected from the group consisting of CLIC1, CLIC2, CLIC3, CLIC4, CLIC5, CLIC6, and combinations thereof, in combination with one or more cannabinoid compounds. The use may comprise administering the transdermal formulation to the mammalian skin.

The present disclosure may also provide for a transdermal formulation for use in treating UV damaged cells and/or preventing UV damage in mammalian skin, the transdermal formulation comprising one or more CLIC proteins, which may be selected from the group consisting of CLIC1, CLIC2, CLIC3, CLIC4, CLIC5, CLIC6, and combinations thereof, in combination with one or more cannabinoid compounds. The use may comprise administering the transdermal formulation to the mammalian skin.

In some embodiments, the amount of CLIC protein is in a therapeutically effective amount. The amount of CLIC protein may be in a range between about 0.0001% to about 10% based on the total weight of the formulation. The CLIC protein can be provided in an amount (as a weight % based on total weight of the formulation) of less than about 40, 35, 30, 25, 20, 15, 10, 5, 2, 1, 0.5, 0.1, 0.05, 0.01 0.005, 0.001, 0.0005 or 0.0001. The CLIC protein can be provided in an amount (as a weight % based on total weight of the formulation) of at least about 0.0001, 0.0005, 0.001, 0.005, 0.01, 0.05, 0.1, 0.5, 1, 2, 5, 10, 15, 20, 25, 30, 35, or 40. The CLIC protein can be in a range provided by any two of these upper and/or lower amounts. In an embodiment, the CLIC protein may be selected from the group consisting of CLIC1, CLIC4, and combinations thereof. In embodiments where two different CLIC proteins are combined, the ratio of the CLIC proteins may be selected from 100:0, 90:10, 80:20, 70:30, 60:40, 50:50, 40:60, 30:70, 20:80, 10:90 or 0:100, as described herein. The ratio of the CLIC proteins can be in a range provided by any two of these values. For example the ratio of CLIC1:CLIC4 may be selected from 100:0, 90:10, 80:20, 70:30, 60:40, 50:50, 40:60, 30:70, 20:80, 10:90 or 0:100. The ratio of CLIC1:CLIC4 can be in a range provided by any two of these values. In some embodiments, the amount of cannabinoid compound is in a therapeutically effective amount. The amount of cannabinoid compound may be in a range between about 0.0001% to about 10% based on the total weight of the formulation. The cannabinoid can be provided in an amount (as a weight % based on total weight of the formulation) of less than about 40, 35, 30, 25, 20, 15, 10, 5, 2, 1, 0.5, 0.1, 0.05, 0.01 0.005, 0.001, 0.0005 or 0.0001. The cannabinoid compound can be provided in an amount (as a weight % based on total weight of the formulation) of at least about 0.0001, 0.0005, 0.001, 0.005, 0.01, 0.05, 0.1, 0.5, 1, 2, 5, 10, 15, 20, 25, 30, 35, or 40. The cannabinoid compound can be in a range provided by any two of these upper and/or lower amounts. In an embodiment, the cannabinoid compound may be selected from cannabidiol (CBD). In an embodiment, the formulation may be in the form of an aqueous solution, alcohol solution, ointment, cream, lotion, gel, paste, salve, spray, controlled-release matrix, transdermal delivery system, patch formulation, or any combination thereof.

It will be appreciated that the dosage regimens and formulations as described herein may apply to any of the embodiments or examples of the methods as described herein.

### Dosage Regimen

As used herein, “therapeutically effective amount” refers to a transdermal formulation as described herein, wherein the one or more CLIC proteins and the one or more cannabinoid compounds is being administered in an amount sufficient to alleviate or prevent to some extent one or more of the symptoms of the condition being treated (e.g. UV damage), typically without undue adverse side effects or to achieve a desired pharmacological effect or therapeutic improvement with a reduced side effect profile. The results can be the reduction and/or alleviation of the signs, symptoms, or causes of a condition, or any other desired alteration of a biological system. In some embodiments, the term “therapeutically effective amount” refers to a formulation as described herein, being administered in an amount sufficient to result in a reduction of symptoms associated UV damage. Therapeutically effective amounts may, for example, be determined by routine experimentation, including but not limited to a dose escalation clinical trial. The phrase “therapeutically effective amount” includes, for example, a prophylactically effective amount. In some embodiments, a prophylactically effective amount is an amount sufficient to prevent UV damage. It is understood that “an effective amount” or “a therapeutically effective amount” can vary from subject to subject, due to variation in metabolism of the compound and any of age, weight, general condition of the subject, the condition being treated, the severity of the condition being treated, and the judgment of the prescribing physician. An appropriate “effective amount” or “a therapeutically effective amount” or “or prophylactically effective amount” in any individual case may be determined by one of ordinary skill in the art using routine experimentation.

The amount of a transdermal formulation as described herein, that will be effective in the treatment and/or prevention of a particular condition disclosed herein will depend on the nature of the condition, and can be determined by standard clinical techniques. In addition, in vitro or in vivo assays may optionally be employed to help identify optimal dosage ranges. Such techniques are known to the person skilled in the art.

The precise dose to be administered to the subject will also depend on the seriousness of the condition, and should be decided according to the judgment of the practitioner and each subject's circumstances.

In some embodiments, the transdermal formulation as described herein, is administered in an amount so as to deliver a total daily dosage (in mg) of at least about 0.0001, 0.0005, 0.001, 0.005, 0.01, 0.05, 0.1, 0.5, 1, 5, 10, 15, 20, 25, 30, 35, 40, 45, 50, 60, 75, 100, 150, or 200 of the one or more CLIC proteins. In some embodiments, the transdermal formulation as described herein, is administered in an amount so as to deliver a total daily dosage (in mg) of less than about 200, 150, 100, 75, 60, 50, 45, 40, 35, 30, 25, 20, 15, 10, 5, 1, 0.5, 0.1, 0.05, 0.01, 0.005 or 0.001 of the one or more CLIC proteins. The total daily dosage may be provided in a range between at any two of these upper and/or lower amounts. For example, a total daily dosage may be provided in an amount of between about 0.0001 and about 100 mg, 0.0001 and about 50 mg, 0.0001 and about 10 mg, 0.0001 and about 5 mg, about 0.001 and about 100 mg, 0.001 and about 50 mg, 0.001 and about 10 mg, 0.001 and about 5 mg, about 0.01 and 100 mg, about 0.01 and 50 mg, about 0.01 and 10 mg, about 0.01 and 5 mg, about 0.1 and 100 mg, about 0.1 and 50 mg, about 0.1 and 10 mg, about 0.1 and 5 mg, about 1 and 100 mg, about 1 and 50 mg, about 1 and 10 mg, about 1 and 5 mg, about 5 and 75 mg, about 10 and 50 mg, about 15 and 45 mg, or about 20 and 40 mg.

In some embodiments, the transdermal formulation as described herein, is administered in an amount so as to deliver a total daily dosage (in mg) of at least about 0.001, 0.005, 0.01, 0.05, 0.1, 0.5, 1, 5, 10, 15, 20, 25, 30, 35, 40, 45, 50, 60, 75, 100, 150, or 200 of the one or more cannabinoid compounds. In some embodiments, the transdermal formulation as described herein, is administered in an amount so as to deliver a total daily dosage (in mg) of less than about 200, 150, 100, 75, 60, 50, 45, 40, 35, 30, 25, 20, 15, 10, 5, 1, 0.5, 0.1, 0.05, 0.01, 0.005 or 0.001 of the one or more cannabinoid compounds. The total daily dosage may be provided in a range between at any two of these upper and/or lower amounts. For example, a total daily dosage may be provided in an amount of between about 0.001 and about 100 mg, 0.001 and about 50 mg, 0.001 and about 10 mg, 0.001 and about 5 mg, about 0.005 and about 100 mg, 0.005 and about 50 mg, 0.001 and about 10 mg, 0.005 and about 5 mg, about 0.01 and 100 mg, about 0.01 and 50 mg, about 0.01 and 10 mg, about 0.01 and 5 mg, about 0.1 and 100 mg, about 0.1 and 50 mg, about 0.1 and 10 mg, about 0.1 and 5 mg, about 1 and 100 mg, about 1 and 50 mg, about 1 and 10 mg, about 1 and 5 mg, about 5 and 75 mg, about 10 and 50 mg, about 15 and 45 mg, or about 20 and 40 mg.

In some embodiments, a therapeutically and/or prophylactically effective amount of a transdermal formulation as described herein, is administered to the subject at a predetermined frequency. In some embodiments, a transdermal formulation as described herein, is administered to the subject according to a dosage regimen in which a transdermal formulation as described herein is administered once daily, twice daily, three times daily, or four times daily. In some embodiments, the transdermal formulation as described herein is administered to the subject according to a dosage regimen in which a transdermal formulation as described herein is administered once daily. In some embodiments, a transdermal formulation as described herein is administered to the subject according to a dosage regimen in which a transdermal formulation as described herein is administered twice daily. In some embodiments, a transdermal formulation as described herein is administered to the subject according to a dosage regimen in which a transdermal formulation as described herein is administered three times daily. In some embodiments, a transdermal formulation as described herein is administered to the subject according to a dosage regimen in which a transdermal formulation as described herein is administered four times daily.

In some embodiments, a transdermal formulation as described herein is administered to the subject according to a dosage regimen in which a transdermal formulation as described herein is administered multiple times daily.

In some embodiments, a therapeutically effective amount of a transdermal formulation as described herein, is administered to the subject at a predetermined frequency and/or duration. For example, administration according to any embodiments (e.g. frequency) as described herein may be for a duration of about, or at least about, 1 day, 1 week, 2 weeks, 3 weeks, 1 month, 2 months, 3 months, 4 months, 5 months, 6 months, 12 months, 2 years, or 5 years. Administration of the therapeutically effective amount of a transdermal formulation as described herein, may be ongoing so long as a therapeutic effect is received by the subject.

As used herein, the term “administer” and “administering” are used to mean introducing the transdermal formulation as described herein, to a subject. When administration is for the purpose of treatment, the transdermal formulation as described herein, is provided at, or after the onset of, a symptom of the particular condition, such as UV damage to cells or mammalian skin. The therapeutic administration of this substance serves to attenuate any symptom, or prevent additional symptoms from arising (e.g. oxidative stress in cells). When administration is for the purposes of preventing or reducing the likelihood of the condition, e.g. oxidative stress in cells, the transdermal formulation as described herein, is provided in advance of any visible or detectable symptom. The prophylactic administration of the transdermal formulation as described herein, serves to attenuate subsequently arising symptoms or prevent or reduce the likelihood of the symptoms from arising altogether.

It will be appreciated by persons skilled in the art that numerous variations and/or modifications may be made to the above-described embodiments, without departing from the broad general scope of the present disclosure. The present embodiments are, therefore, to be considered in all respects as illustrative and not restrictive.

### EXAMPLES

The present disclosure is further described by the following examples. It is to be understood that the following description is for the purpose of describing particular examples only and is not intended to be limiting with respect to the above description.

### Example 1 General Processes and Preparation

### Example 1a Cell Culture

The following reagents were purchased from ThermoFisher: Human Fibroblast Expansion Basal Medium, Low Serum Growth Supplement (LSGS), EpiLife™ Medium with 60 μM calcium, Human Keratinocyte Growth Supplement (HKGS), Trypsin-EDTA (0.05%), phenol red. Fetal Bovine Serum (French Origin) was purchased from Scientifix. Human skin cells: Fibroblasts (HDF) and Keratinocyte (HEK) cells were purchased from Sigma Aldrich and ThermoFisher respectively. HDF cells were grown in Fibroblast media supplemented with 1×LSGS, while HEK cells were grown in Keratinocyte media supplemented with 1×HKGS. Both cell lines were kept in an incubator at 37° C., with 5% CO₂.

The human skin cells (HDF and HEK) were cultured in respective media and were grown in T25 flasks until ~70-80% confluent. Once confluent, the cells were washed with 1×PBS and split using 0.05% Trypsin-EDTA solution. The cell pellet was collected and split into a fresh T25 flask and T75 flask containing full growth media at a dilution factor of 1:4 and incubated at 37° C., 5% CO₂. Cells were >85-92% viable based on Trypan blue exclusion when seeding and passing.

On the day of the experiment, the human skin cells were washed, lifted, and seeded at a density of 20,000 cells per well for HDF cells and 40,000 cells per well for HEK cells on a clear 96 well plate and incubated overnight prior to treatment with CLIC proteins followed by treatment with oxidizing agents.

### Example 1b Protein Purification of rCLIC1 and rCLIC4

### (i) 2×YT Media for Bacterial Growth:

Standard microbial growth 2×YT medium was prepared using yeast extract (10 g), bacteriological tryptone (15 g) and NaCl (5 g) (Sigma Aldrich). The materials were dissolved in sterile water and brought to a final volume of 1 liter followed by autoclaving.

(ii) Culture Preparation, Harvesting and Lysing the E. coli:

Glycerol stocks of E. coli BL21 (DE3) cells were transformed with the His-tagged pET28 (+) expression vector (Novagen) containing the coding sequence for either human CLIC1 or CLIC3. The human CLIC4 was prepared in the pGEX4T-1 vector (AMRAD-Pharmacia). This vector coded for an N-terminal GST purification tag. The bacteria were inoculated in sterile conical flasks containing 20 ml of 2×YT media with 20 ul kanamycin at a concentration of 30 μg/ml (Sigma Aldrich) or 100 μg/ml ampicillin in the case of CLIC4. The cells were left to grow overnight at 37° C. with 200 rpm shaking. Small scale bacteria cultures were upscaled into large flasks containing 350 ml 2×YT media with either 30 μg/ml kanamycin or 100 μg/ml ampicillin and left to grow at 37° C. with 200 rpm shaking for 1.5 hours. Once the bacteria achieved an optical density (OD) at 600 nm between 0.6-0.8, the cells were induced with 1 mM of IPTG (Isopropylthiogalactoside) at 20° C. with 200 rpm shaking for 16 hours. The induced cells were harvested by centrifugation using the Hitachi high-speed centrifuged equipped with a R13A rotor at 10600 rpm for 30 minutes at 4° C. The bacterial pellets were collected, pooled, and resuspended in 10 ml lysing buffer (300 mM NaCl, 50 mM potassium phosphate buffer pH 8.0 and 5 mM Imidazole) or resuspended in phosphate buffered saline containing 0.5 mM TCEP (CLIC4) and then sonicated for 15-20 second cycles at 700 psi (Sonics & Materials Company/Vibra-Cell Ultrasonic Liquid Processors). Afterwards, 1 ml 20% TritonX-100 solution was added to the 10-15 ml bacterial homogenates. The soluble cell lysates were then collected after an additional centrifugation at 12,400 rpm for 40 minutes at 4° C. using the Hitachi high-speed centrifuged equipped with a R18A rotor.

(iii) Enzymatic Cleavage of Thrombin Followed by Size Exclusion Chromatography:

The soluble fractions of the cell lysates were subjected to affinity chromotography using either a Ni2+-NTA (Qiagen) column for CLIC1 or CLIC3 proteins or a GSTrap 4B (GE Healthcare) column for CLIC4. The resin slurry was loaded into an affinity column and allowed to settle, afterwards it was washed via gravity flow with 10 mL of wash buffer 1 (5 mM Imidazole, 300 mM KCl, 50 mM potassium phosphate buffer pH 8, 0.5 mM TCEP) and then 10 ml of wash buffer 2 (10 mM Imidazole, 300 mM KCl, 50 mM potassium phosphate buffer pH 8, 0.5 mM TCEP). The cell lysates were added to their respective columns and incubated at 4° C. for 1 hour prior being washed thrice with wash buffer 1 and wash buffer 2 to remove any unwanted unbound proteins. The His-tag or the GST-tag was removed by in column thrombin enzymatic cleavage using an overnight incubation of bovine plasma thrombin (Sigma Aldrich) (30 NIH units per 1 L of bacterial culture) at 4° C. The cleaved CLIC proteins were then collected in PBS buffer (10 mM phosphate buffer, 2.7 mM KCl, 140 mM NaCl, pH 7.4, and 0.5 mM TCEP). The proteins were further purified through size exclusion chromatography (SEC) (AKTA Pure/Amersham Pharmacia Biotech) using a HiPrep™ 16/60 Sephacryl® S-100 HR (Sigma Aldrich) for CLIC1/CLIC3 or a HiLoad 16/600 Superdex 75 pg (GE Healthcare) column for CLIC4 and equilibrated in column sizing buffer (100 mM KCl, 1 mM NaN3, 20 mM HEPES pH 7.5 and 0.5 mM TCEP). The purified samples were stored at −80° C.

Recombinant protein concentrations were measured spectrophotometrically and calculated using the following extinction coefficient values of 0.647,0.391 and 0.745 for CLIC1, CLIC3 and CLIC4 respectively by the Bradford Protein Assay Kit (ThermoFisher Scientific) according to the manufacturer's instructions. Afterwards, SDS-PAGE was run using 4-15% Mini-PROTEAN® TGX Stain-Free™ Protein Gels (Bio-Rad, Australia) and visualized via Coomassie brilliant blue staining (Bio-Rad).

(iv) Endotoxin Removal and Treatment of Cells with Recombinant CLIC:

Before adding the recombinant CLIC1 and CLIC4 proteins directly to the human skin cells, the Pierce High-Capacity Endotoxin Removal Spin Column (ThermoFisher Scientific) was used. After endotoxin removal, the samples were quantified using the Pierce™ BCA Protein Assay Kit (ThermoFisher Scientific) and the purity of the recombinant proteins were verified by SDS-PAGE and Dot-blot Analysis.

Dot blot analysis using anti-CLIC antibodies against their respective CLIC proteins (data not shown) confirmed the presence of the desired protein samples. In addition to dot blot, SDS-PAGE analysis confirmed the successful cleavage of 6× Histidine-tag off CLIC protein, thus resulting in a purity of approximately 90-95%. Notably, no degradation products were detected by the absence of other protein bands in the SDS-PAGE gel (data not shown). Protein concentration was then determined by Bradford assay for each of the proteins purified. The purified protein fractions were then aliquoted and stored at −80° C. for future use and were also subjected to functional analysis.

### Example 1c Enzyme Assay-HEDS (2-Hydroxyethyl Disulphide)

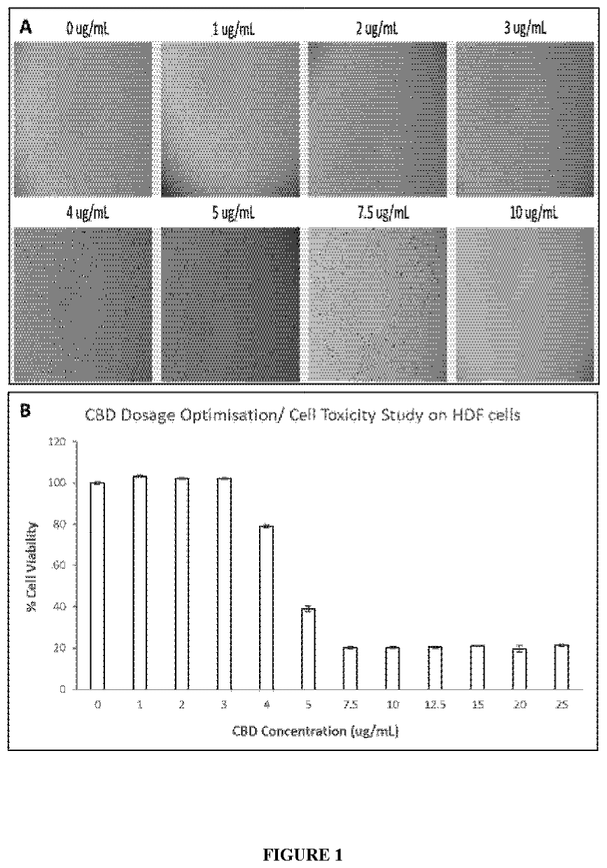

The functional activity of the purified CLIC1, CLIC3 and CLIC4 proteins were analysed using the HEDS assay. The assay measures the ability of the CLIC proteins to reduce the HEDS substrate when coupled with glutathione (GSH) and glutathione reductase (GR) resulting in the consumption of NADPH that is monitored at A340 nm. As seen in FIG. 1, the buffer control sample containing all the reagents, except for CLIC proteins, shows no enzymatic activity over time. However, addition of the different CLIC proteins to the reaction mixture results in a decrease in the absorbance at 340 nm, that in turn correlates to the consumption of NADPH indicating significant enzymatic activity.

### Example 1d Preparation of Cannabinoid Compound-Cannabidiol (CBD)

Stock solution of CBD was prepared by first dissolving 100 mg of the soluble form of CBD in 50 mL of just-boiled water with continuous stirring at 600 rpm on a magnetic stirrer to form a homogenous suspension followed by gradual addition of 200 mL room temperature water to the suspension whilst continuing to stir. This stock solution was then used to prepare 3 ug/mL CBD solution in HDF or HEK media. CLIC proteins were added in different CLIC1:CLIC4 ratios to either HDF or HEK media containing 3 ug/mL CBD solutions in order to prepare the CBD-CLIC combinations. Total CLIC protein concentrations were maintained at a final CLIC1 concentration=3 ug/mL and CLIC4=5 ug/mL.

### Example 1e Production of Unilamellar Liposomes

Phospholipids are dissolved in chloroform and dried over nitrogen gas overnight to prepare lipid cakes to which sterile phosphate buffer saline (PBS) (pH 7.4) is added and sonicated to form the liposomes.

Liposomes are then washed twice with PBS by centrifugation at 12000 rpm and resuspended in PBS buffer. The lipid suspension is then passed through an Avati Polar Extrusion to form uniformly sized, unilamellar vesicles.

CLIC proteins and CBD can then be added to the liposomes to give a final concentration of 1 mg/ml protein. This liposome/protein suspension can then be added to cells as required and assays performed. Stability testing may also performed with these liposomes alone or in combination with CLIC and CBD over a period of time at different temperatures in different biological buffers.

### Example 2 Effects of rCLIC1, rCLIC4 and CBD on the Viability of Human Skin Cells Challenged with H₂O₂ and UV

Example 2 describes a study performed using human skin fibroblast (HDF) and keratinocyte (HEK) cells to assess the effectiveness of purified recombinant CLIC proteins in combination with CBD to provide protection to these cells when challenged with the oxidative stressor, hydrogen peroxide, and UV exposure.

To determine the optimal concentration of CBD to use for cell protection and anti-oxidant activity studies, cell toxicity assay was performed where varying concentration of CBD ranging from 0 g/mL to 25 μg/mL was added to HDF cells and incubated for 24 hours. FIG. 1A shows the HDF cell images at varying CBD concentration and FIG. 1B shows the % cell proliferation and viability of HDF cells measured using WST-1 reagent assay following 24 hours incubation with varying concentrations of CBD. As seen in FIG. 1, CBD concentrations above 4 μg/mL are toxic to human fibroblast cells.

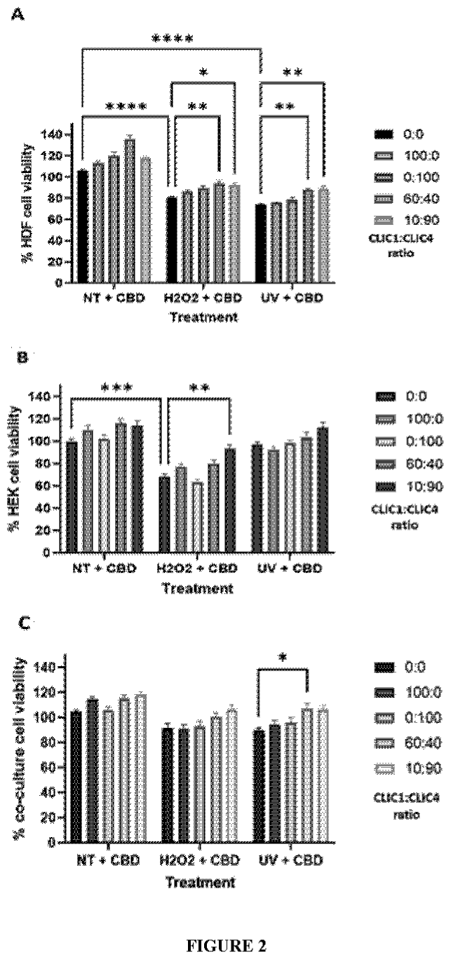

The effect of individual proteins CLIC1 or CLIC4 or combinations of CLIC1:CLIC4 in the presence of CBD were tested on HDF, HEK and Co-cultured cells and the results are shown in FIG. 2 (A, B and C) respectively. Varying dosage ratios of CLIC1:CLIC4 proteins in combination with 3 ug/mL CBD were added extraneously to HDF, HEK or co-cultured cells 24 hours prior to the cells being subjected to oxidative stress either via treatment with 0.2 mM H₂O₂ for 1 hour or UV exposure [2.175×10¹³ (J*m⁻²*sec⁻¹)] for a period of 20 minutes in order to induce 25% cell damage.

FIG. 2 shows the percentage cell viability of HDF (FIG. 2A), HEK cells (FIG. 2B) or co-cultured cells (FIG. 2C) treated with CBD plus varying combinations of CLIC1:CLIC4. Cells treated with CBD plus different CLIC1:CLIC4 ratios but not exposed to UV or H₂O₂ (NT+CBD) and cells treated with 3 ug/mL CBD but no CLICs (0:0) were used as controls. As seen in FIG. 2, the presence of CBD plus CLIC1:CLIC4 proteins in varying ratios all showed protection against H₂O₂ and UV damage in HDF (FIG. 2A) or HEK (FIG. 2B) cells. The combinations of CBD with CLIC1:CLIC4 ratios of 60:40 and 10:90 showed significantly greater amount of protection in comparison to CBD with either of the CLICs alone. This data clearly shows the protective effects of the CBD with combinations of CLIC1:CLIC4 against H₂O₂ and UV damage when added extraneously to human skin cells. Similar results were also obtained using co-cultured HDF and HEK cells (FIG. 2C) where the CLIC1:CLIC4 ratios of 60:40 and 10:90 showed greater amount of protection in comparison to CLICs alone in the presence of CBD when added extraneously to co-cultured cells.

### Example 3 Further Studies on the Effects of CLIC1, CLIC4 and CBD on the Viability of Human Skin Cells Challenged with H₂O₂ and UV

Example 3 describes a further study performed using human skin fibroblast (HDF) and keratinocyte (HEK) cells to assess the effectiveness of CLIC proteins in combination with CBD to provide protection to these cells when challenged with the oxidative stressor, hydrogen peroxide, and UV exposure

HDF and HEK cells were seeded alone in 96 well plates or co-cultured models were achieved by growing the HEK cells on top of 70% confluent HDF cells grown over a period of two days. The cells were then treated for 24 hours with either CLIC1 or CLIC4 alone or in combination of CLIC1:CLIC4 ratios of 60:40 in the absence or presence of Aquaphase CBD (Aq-CBD). Aquaphase CBD can be prepared as described in patent application WO 2007/093177, in particular page 30 line 30 to page 31 line 2. The cells were then subjected to oxidative stress following treatment with either 0.2 mM H₂O₂ for 1 hour or exposed to 20 mins UV radiation. Cell proliferation and viability assays were then conducted to determine the anti-oxidant and cell protection ability of the exogenously added CLIC proteins. Data was collected from three different passage numbers and each passage was run in triplicate for each of the different cell types (n=9 for each cell type)

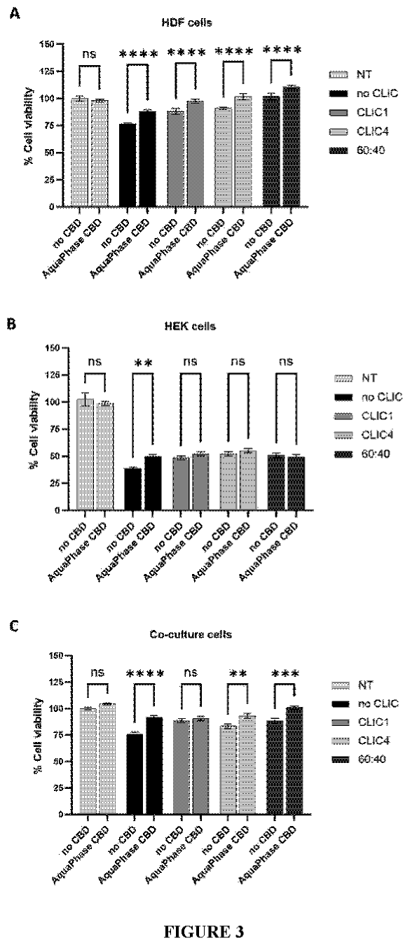

FIG. 3 shows the percentage cell viability of HDF (FIG. 3A), HEK cells (FIG. 3B) or co-cultured cells (FIG. 3C) treated with or without Aq-CBD in varying combinations with CLIC proteins for 24 hours before being subjected to 1 hour H₂O₂ treatment. Cells not treated with CLICs or H₂O₂ (NT) and cells treated with 3 ug/mL CBD but no CLICs (no CLIC) were used as controls. As seen in FIG. 3, the presence of CBD with CLIC1:CLIC4 proteins in varying ratios all showed significant protection against H₂O₂ in HDF (FIG. 3A) and co-culture cells (FIG. 3C). The combinations of CBD with CLIC1:CLIC4 ratio of 60:40 showed significantly greater amount of protection in comparison to CBD with either of the CLICs alone or by itself. This data shows the protective effects of CBD with combinations of CLIC1:CLIC4 against H₂O₂ when added extraneously to human skin cells, indicating a synergistic protective effect between the CLICs and CBD. Similar results were obtained using co-cultured HDF cells (FIG. 3C) where the CLIC1:CLIC4 ratios of 60:40 showed greater protection in comparison to CLICs alone or in the presence of CBD when added extraneously to co-cultured cells. No synergistic effect between the CLICs and Aq-CBD was observed in the human keratinocyte cells treated with hydrogen peroxide (FIG. 3B).

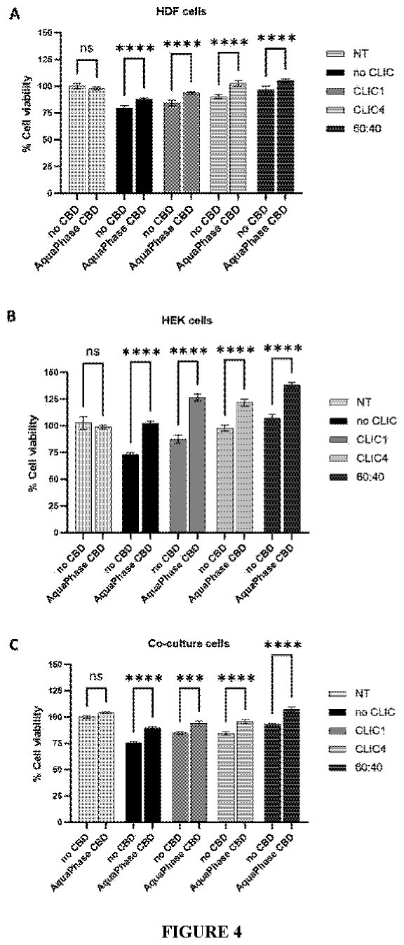

FIG. 4 shows the percentage cell viability of HDF (FIG. 4A), HEK cells (FIG. 4B) or co-cultured cells (FIG. 4C) treated with or without Aq-CBD in varying combinations with CLIC proteins for 24 hours before being subjected to 20 minutes of UV exposure. Cells not treated with CLICs or UV (NT) and cells treated with 3 ug/mL CBD but no CLICs (no CLIC) were used as controls. As seen in FIG. 4, the presence of CBD with CLIC1:CLIC4 proteins in varying ratios all showed significant protection against UV exposure in HDF (FIG. 4A), HEK (FIG. 4B) and co-culture cells (FIG. 4C). The combinations of CBD with CLIC1:CLIC4 ratio of 60:40 showed significantly greater amount of protection in comparison to CBD with either of the CLICs alone or by itself in all the different cell types. This data shows the protective effects of the CBD with combinations of CLIC1:CLIC4 against UV when added extraneously to human skin cells, indicating a synergistic protective effect between the CLICs and CBD.

1. A transdermal formulation comprising one or more CLIC proteins and one or more cannabinoid compounds, in a therapeutically and/or prophylactically effective amount, and a carrier.

2. The formulation of claim 1, wherein the CLIC protein is in an amount between about 0.0001% and 10% based on total weight of the formulation.

3. The formulation of claim 1, wherein the CLIC protein is CLIC1, CLIC2, CLIC3, CLIC4, CLIC5, CLIC6, or a combination thereof, preferably CLIC1 or CLIC4.

4. (canceled)

5. The formulation of claim 1, wherein the one or more cannabinoid compounds is cannabidiol (CBD), cannabidiol A (CBDA) cannabigerol (CBG), cannabichromene (CBC), cannabicyclol (CBL), cannabinol (CBN), cannabigerolic acid (CBGA), cannabidivarin (CBDV), cannabichromevarin (CBCV), cannabigerovarin (CBGV), cannabigerol monomethyl ether (CBGM), or a combination thereof, preferably CBD.

6. (canceled)

7. The formulation of claim 1, wherein the cannabinoid compound is present in the form of a cannabinoid extract or isolate having less than 1, 0.5, or 0.3 wt % of any of tetrahydrocannabinol (THC), tetrahydrocannabivarin (THCV) and tetrahydrocannabinolic acid (THCA), and/or wherein the formulation comprises the one or more cannabinoid compounds in a concentration of between about 0.0001 mg/ml to about 1200 mg/ml.

8. (canceled)

9. The formulation of claim 1, wherein the carrier is a aqueous carrier.

10. The formulation of claim 1, wherein the solvent carrier is water, an alcohol, a lanolin alcohol, a farry alcohol, a glycol, an oil, a wax, a phospholipid, a sterol, isopropyl myristate, oleic acid, or a combination thereof, optionally wherein the solvent carrier is in an amount between about 2% and 99.99% based on total weight of the formulation.

11. (canceled)

12. The formulation of claim 1, wherein the formulation comprises one or more additives, optionally selected from antioxidant, preservative, salt, sugar, sugar alcohol, polysaccharide, vitamin, essential oil, rheology modifier, transdermal compounding base or gel, lipoic acid, xanthan gum, surfactant, or a combination thereof.

13. (canceled)

14. The formulation of claim 12, wherein the additive is present in an amount between about 0.01% and 15% based on total weight of the formulation.

15. The formulation of claim 1, wherein the formulation comprises a penetration enhancer, optionally wherein the penetration enhancer is water, sulphoxide, azone, pyrrolidone, alcohol, alkanol, glycol, surfactant, fatty acid, terpene, terpenoid, or a combination thereof.

16. (canceled)

17. The formulation of claim 15, wherein the penetration enhancer is present in an amount between about 0.01% and 15% based on total weight of the formulation.

18. The formulation of claim 1, wherein the formulation further comprises a buffer comprising glycerol, glutathione, phosphate-based buffer, acetate buffer, phosphate buffer, citrate buffer, TRIS buffer, maleate buffer, succinate buffer, histidine buffer, amino acids, or a combination thereof, optionally wherein the buffer is in an amount between about 0.01% and 15% based on total weight of the formulation.

19. (canceled)

20. The formulation of claim 1, wherein the formulation is in the form of an aqueous solution, alcohol solution, ointment, cream, lotion, gel, paste, salve, spray, controlled-release matrix, transdermal delivery system, patch formulation, or any combination thereof.

21. A method of reducing and/or preventing oxidative stress in cells of mammalian skin, the method comprising: topically administering a transdermal formulation to the mammalian skin, wherein the transdermal formulation comprises a therapeutically and/or prophylactically effective amount of one or more CLIC proteins, one or more cannabinoid compounds, and a carrier.

22. (canceled)

23. A method of treating or rejuvenating UV damaged cells and/or preventing UV damage in mammalian skin, the method comprising: topically administering a transdermal formulation to the mammalian skin, wherein the transdermal formulation comprises a therapeutically and/or prophylactically effective amount of one or more CLIC proteins, one or more cannabinoid compound compounds, and a carrier.

24. The method of claim 23, comprising steps of administering the formulation topically in the form of an ointment, cream, lotion, gel, salve, spray, controlled-release matrix, transdermal delivery system, patch formulation, or any combination thereof.

25. The method of claim 23, comprising steps of administering the formulation over a time period of about 1 day to about 6 months.

26. The method of claim 23, comprising steps of administering the formulation in a dosage of the one or more CLIC proteins in a range between about 0.0001 mg to about 100 mg per day and the one or more cannabinoid compounds in a range between about 0.001 mg to about 100 mg per day.

27. The method of claim 23, comprising steps of administering the formulation one, two, three or four times per day.

28-40. (canceled)
